(19) United States
(12) Plant Patent Application Publication
(10) Pub. No.: US 2025/0212708 P1
(43) Pub. Date: Jun. 26, 2025

(54) CACAO VARIETY 'MINI'

(50) ;

(71) Applicant: Mars, Incorporated, McLean, VA (US)

(72) Inventor: Ashley DUVAL, Davis, CA (US)

(73) Assignee: Mars, Incorporated, McLean, VA (US)

(21) Appl. No.: 18/991,066

(22) Filed: Dec. 20, 2024

Related U.S. Application Data
(60) Provisional application No. 63/614,448, filed on Dec. 22, 2023.

(51) Int. Cl. A01H 6/00; A01H 5/08

(52) U.S. Cl. CPC A01H 6/00; A01H 5/08
USPC PLT/152

(57) ABSTRACT

A new and distinct variety of cacao plant designated as ‘Mini’, particularly selected for its size, leaf structure, coloring, and self-compatibility, is disclosed.

### CROSS-REFERENCE TO RELATED APPLICATION

This application claims the benefit of U.S. Provisional Application No. 63/614,448,filed Dec. 22, 2023, which is hereby incorporated by reference in its entirety.

Latin name: Botanical classification: Theobroma cacao L.

Varietal denomination: Variety denomination: the varietal denomination of the cacao variety disclosed herein is ‘Mini’.

### BACKGROUND OF THE INVENTION

Cacao beans are produced by cacao trees which are found in warm, moist climates in areas about 20 degrees latitude north and south of the Equator. The cacao tree produces leaves, flowers and fruit throughout the year, and the ripe fruit or pod resembles a long cantaloupe, typically containing from about 20 to 40 almond-shaped cacao beans. Cacao butter and cacao solids are extracted from commercial cacao beans and processed to produce various cacao and chocolate products. Different varieties of cacao trees have varying cacao bean yield, resistance to disease, and levels of compounds that impact the flavor and nutritional value of cacao products. Cacao is an important crop that has been appreciated by humans for thousands of years.

Accordingly, there is a need for new varieties of cacao plants. In particular, there is a need for improved varieties of cacao plants that possess novel aesthetic characteristics and are stable and suitable for cultivation in various environments, such as, for example, in an at-home ornamental setting.

### SUMMARY OF THE INVENTION

In order to meet these needs, the present invention is directed to an improved variety of cacao plant. In particular, the present invention relates to a new and distinctive variety of cacao plant (Theobroma cacao L) that has been denominated ‘Mini’. Cacao variety ‘Mini’ has attractive foliage with colorful flushing and interesting leaf texture, a thick cuticle providing improved resistance to pests and wind damage, small height and upright growth, slow growth requiring low amounts of water, and small and colorful pods and flowers. Variety ‘Mini’ is characterized by its self-compatibility and ability of yielding both flower and fruit while maintaining dwarf stature.

Cacao variety ‘Mini’ was developed from a controlled cross between the cacao female parent ‘CCN-51’ (unpatented) and the cacao male parent ‘Crinkle Leaf’ (unpatented) in 2018, which produced an F1 population of 248 individuals. Clonal selections were made from this F1 population based on criteria of aesthetic, ornamental features, ease of propagation, and pod traits. Additional seedling selections were then made from F2 individuals for further breeding and development of desirable traits. Cacao variety ‘Mini’ is a specific selection from this F2 population. Cacao variety ‘Mini’ was asexually propagated via grafting onto rootstock in Davis, California, USA.

The cacao variety ‘Mini’ is characterized by shortened internodes, and small crispate leaves with a stiff and highly rugose surface. Growth of plagiotropic shoots is fastigiate, secondary branching is reduced, and branches and petioles are thicker and more rigid than standard cacao varieties. Floral parts are reduced and have a rounder, wider appearance than other cacao varieties. Cacao variety ‘Mini’ shows segregation of pigmentation in the F1 new flushes, flowers and pods. Variation in the pigmentation of pedicels ranges from green to blush in cacao variety ‘Mini’.

Cacao variety ‘Mini’ comprises a mutation inherited from the paternal clone, ‘Crinkle Leaf’, that contributes to shortened internodes, and small crispate leaves with a stiff and highly rugose surface. Growth of plagiotropic shoots is fastigiate, branches and petioles are thicker and less malleable than in wild type cacao, and floral parts are reduced and have a rounder, wider appearance. The traits of cacao variety ‘Mini’ may vary within a range depending on the environmental conditions. For example, the coloration of the leaf flush may vary over time and/or with variations in the nutrients in the soil.

Mutations inherited from the maternal clone, ‘CCN-51’, contribute to segregation of pigmentation in the Fl (e.g. F1 progeny of ‘Mini’ self-pollination) new flushes, flowers and pods, longer and larger pods, and higher fructification.

Cacao variety ‘Mini’ flowers are characterized by self-compatibility, longer and larger pods with more seeds than the parent ‘Crinkle Leaf’, and higher fructification. Seeds range from dark purple to pink and mottled, and show two distinct morphotypes within a pod: wide and flat or reduced and round. Additionally, ˜25% of the seeds produced from self-pollinations of Cacao variety ‘Mini’ are extremely reduced such that the bean appears empty, with shortened and rounded cotyledon and root radical, and apparently non-viable.

The mutation contributing to the distinctive habit and foliar characteristics of Cacao variety ‘Mini’ are controlled by a dominant allele at a single locus acting in a pleiotropic manner, with the trait segregating in a 1:1 ratio.

‘Mini’ was particularly selected for its size, leaf structure, coloring, and self-compatibility.

### BRIEF DESCRIPTION OF THE DRAWINGS

This new cacao plants are illustrated by the accompanying photographs. The colors shown are as true as can be reasonably obtained by conventional photographic procedures.

FIGS. 1A-1O illustrate whole plants of variety ‘Mini’. FIGS. 1A-1C, FIG. 1I, and FIGS. 1K-1O illustrate individual, whole plants of variety ‘Mini’, both as seedlings and grafted plants. FIGS. 1D-1G and FIG. 1J illustrate whole plants of variety ‘Mini’ that are bearing fruit. FIG. 1H illustrates a whole plant of variety ‘Mini’ with leaves that display a range of colors.

FIGS. 2A-2Q illustrate shoots and leaves of variety ‘Mini’. FIG. 2A illustrates a shoot of variety ‘Mini’ with attached, alternating leaves. FIG. 2B illustrates many developing buds along a shoot. FIG. 2C illustrates leaves on diverging nodes of variety ‘Mini’. FIGS. 2D-2H illustrate leaves of variety ‘Mini’. FIGS. 2I-2Q illustrate the striking range of colors exhibited by leaves of variety ‘Mini’, wherein leaves are initially dark red and become greener as they mature.

FIGS. 3A-3H illustrate exemplary flowers of variety ‘Mini’. FIG. 3A illustrates variety ‘Mini’ flowers across a developmental series, from bud to open flower. FIG. 3B illustrates nodes on variety ‘Mini’ shoots that bear inflorescences. FIG. 3C illustrates an open and a closed flower of variety ‘Mini’. FIGS. 3D-3G illustrate open flowers of variety ‘Mini’. FIG. 3H illustrates an open flower of the paternal variety ‘Crinkle Leaf’.

FIGS. 4A-4Y illustrate fruit of variety ‘Mini’. FIGS. 4A-4N and FIGS. 4S-4V illustrate the color, shape, and surface texture of multiple fruit of variety ‘Mini’. FIGS. 4O-4P and FIG. 4W illustrate cross-sections of variety ‘Mini’ fruit wherein the pulp and seeds have been removed. FIGS. 4Q-4R illustrate cross-sections of variety ‘Mini’ fruit wherein the pulp and seeds are intact. FIG. 4X illustrates pods of the paternal variety ‘Crinkle Leaf’, and FIG. 4Y illustrates cross sections of the paternal variety ‘Crinkle Leaf’.

FIGS. 5A-5C illustrate exemplary seeds of variety ‘Mini’.

### DETAILED BOTANICAL DESCRIPTION

The following description is based on observations taken of plants and fruits of the new and distinctive cacao variety of the present invention.

Cacao variety ‘Mini’ has shown uniformity and stability for its traits, within the limits of environmental influence for the traits. Cacao variety ‘Mini’ has been increased with continued observation for uniformity. No variant traits have been observed or are expected in ‘Mini’.

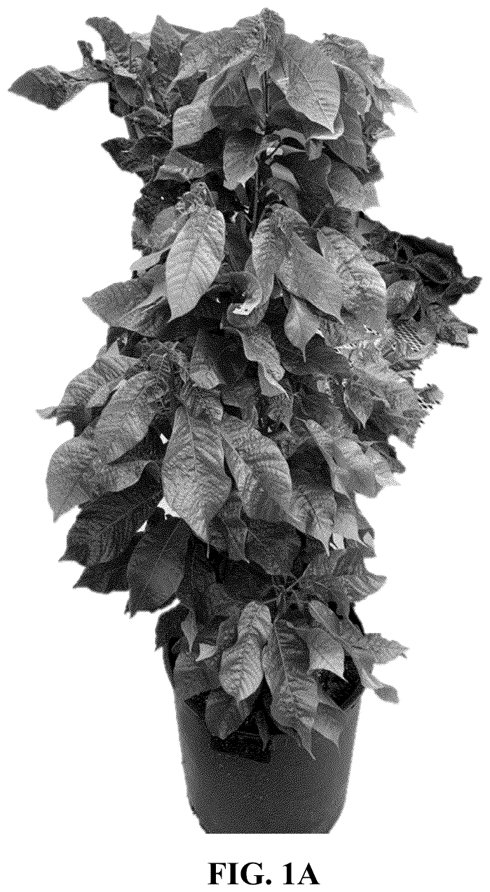

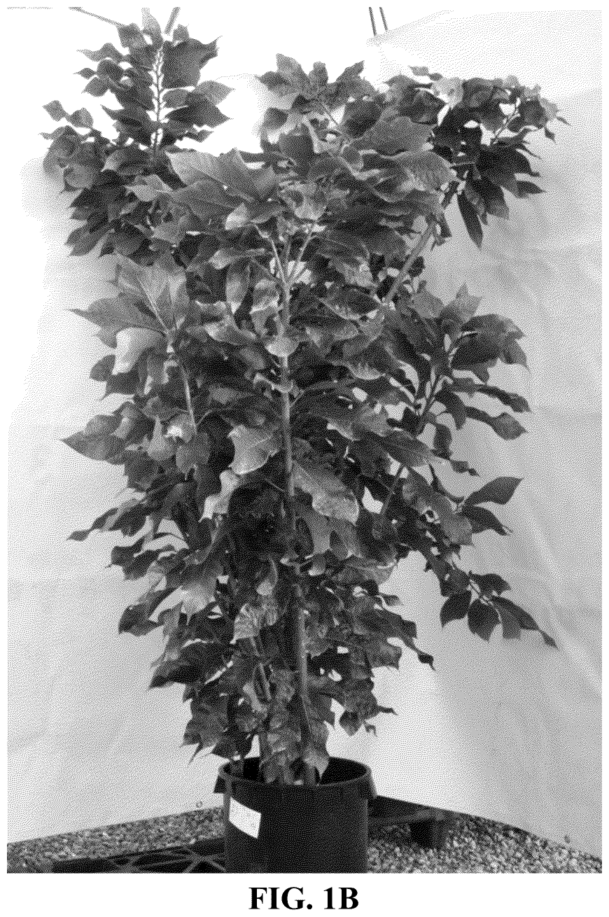

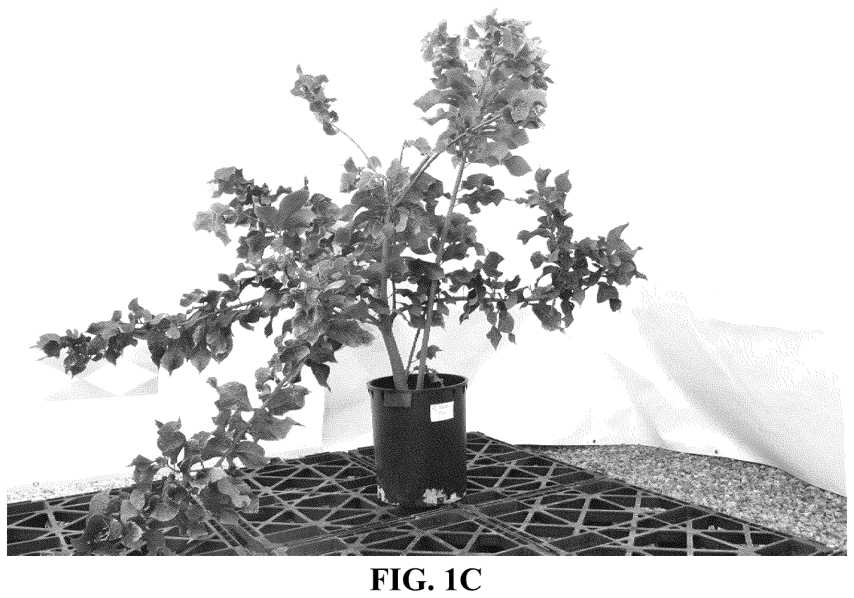

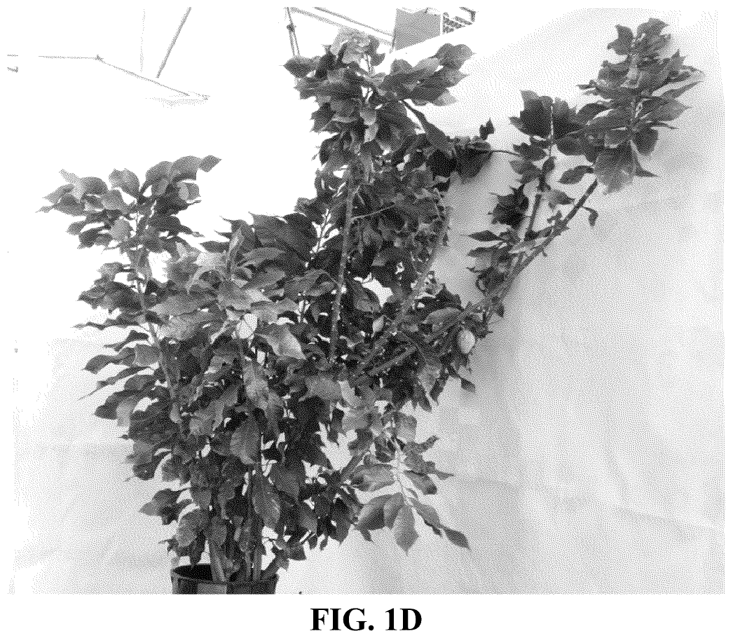

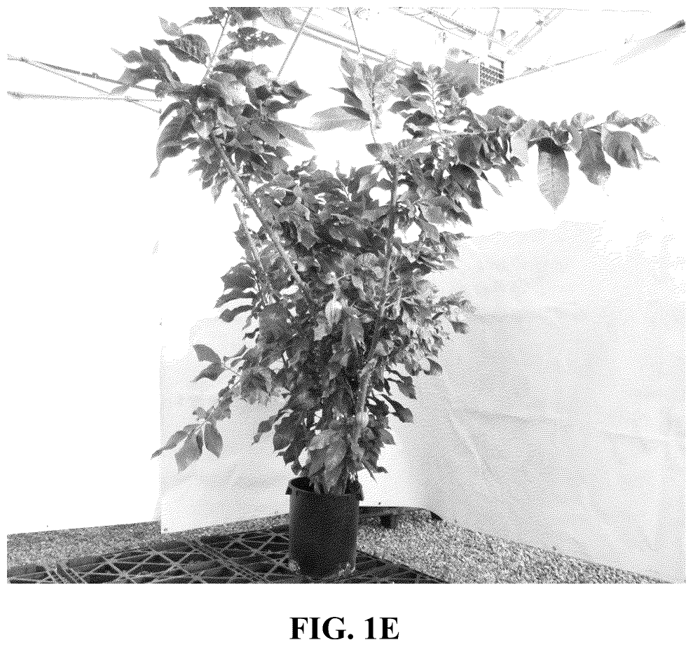

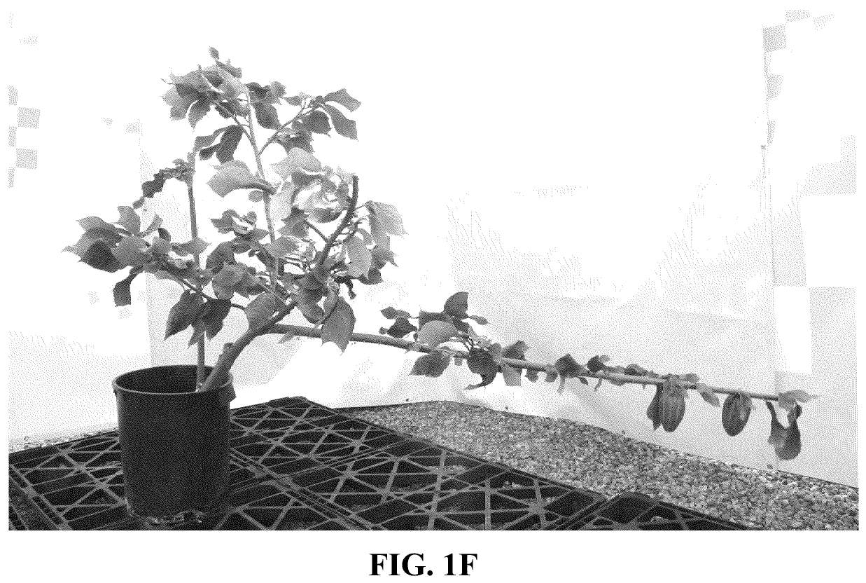

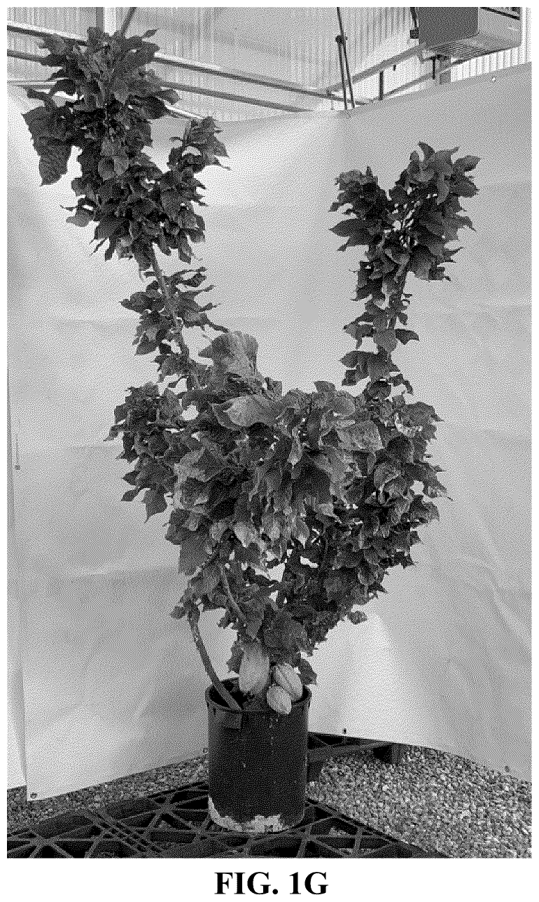

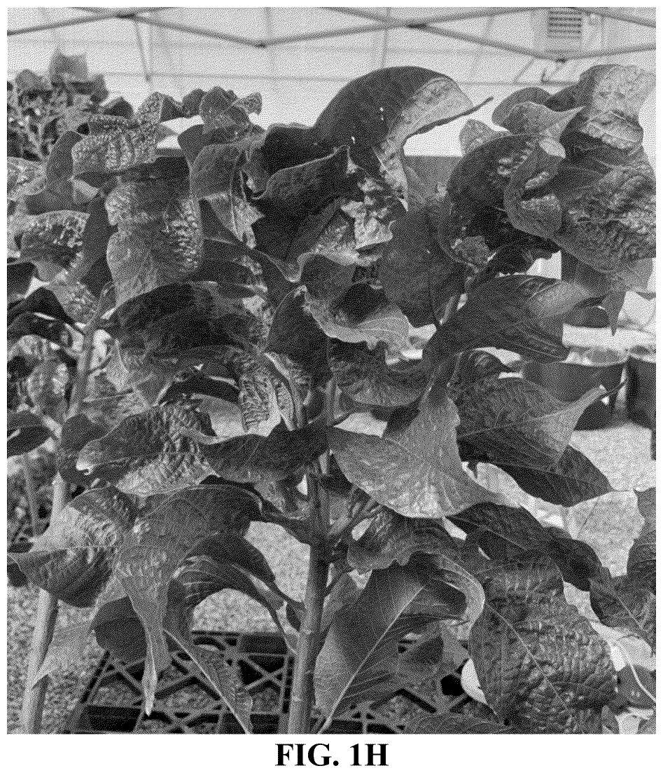

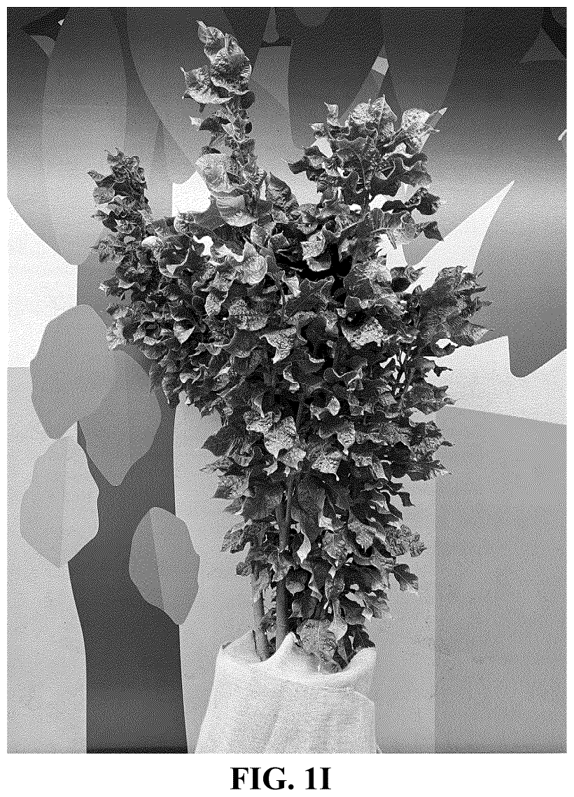

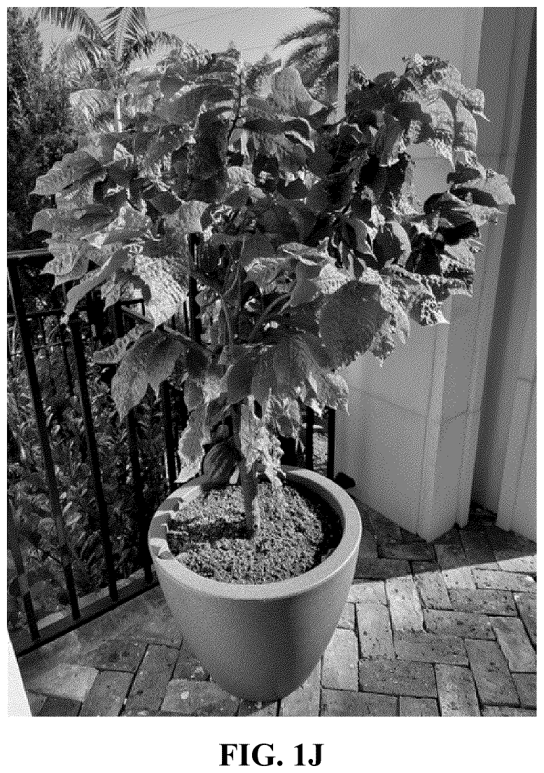

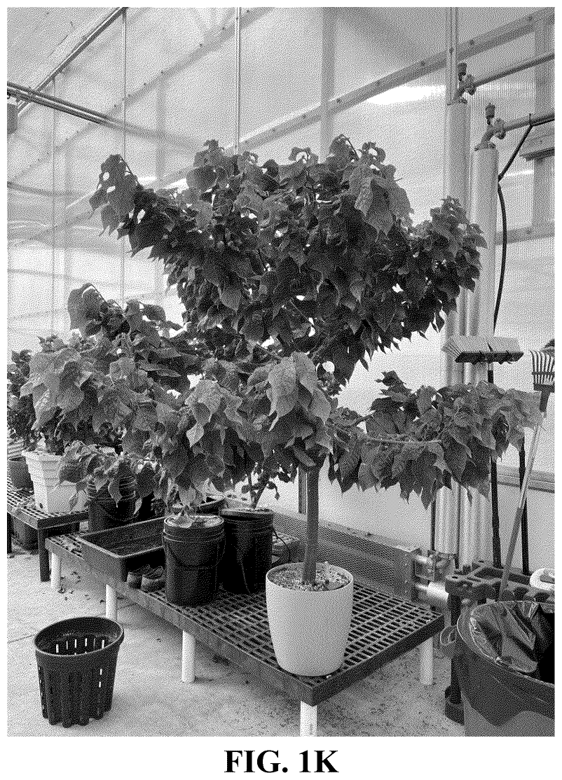

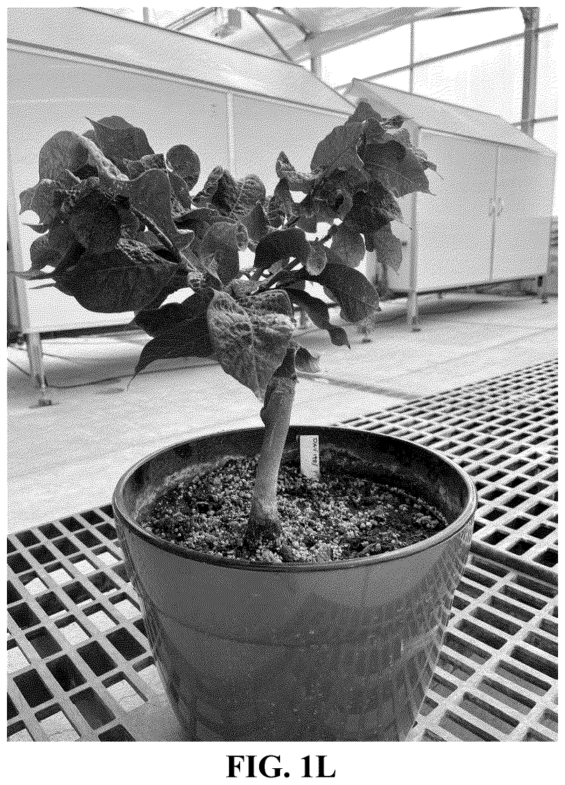

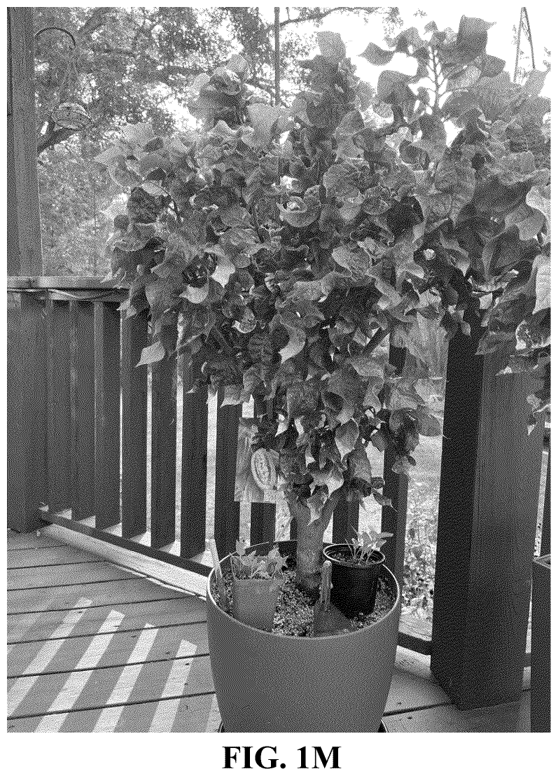

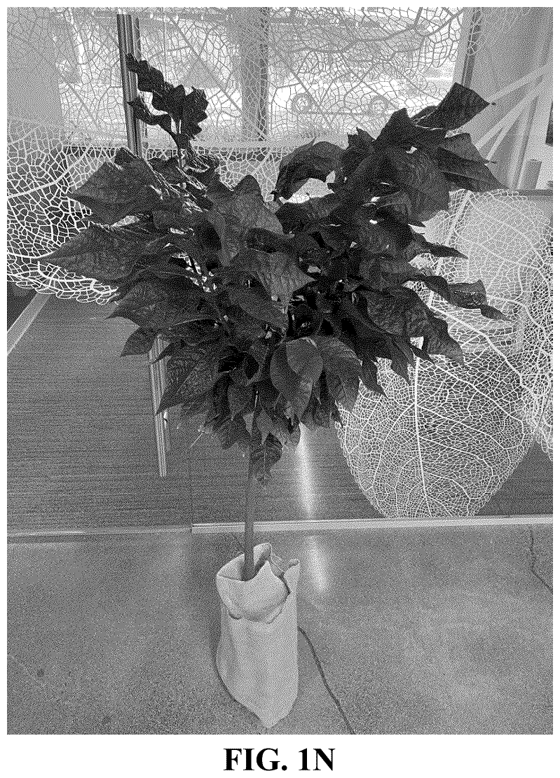

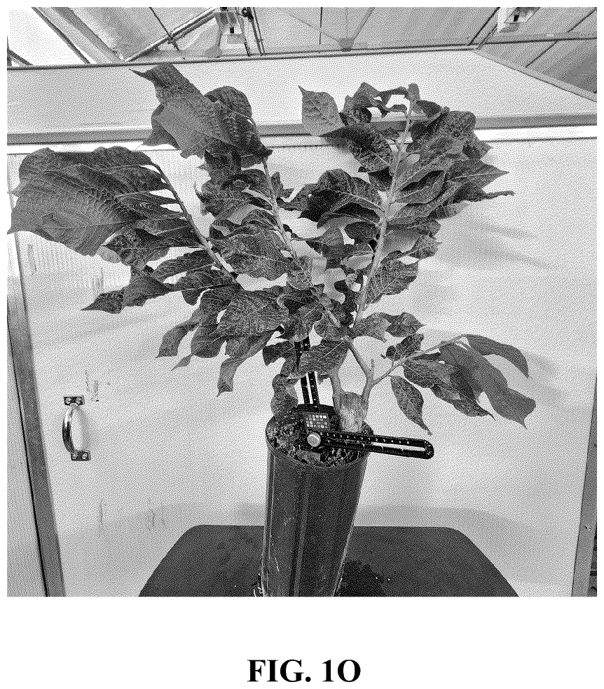

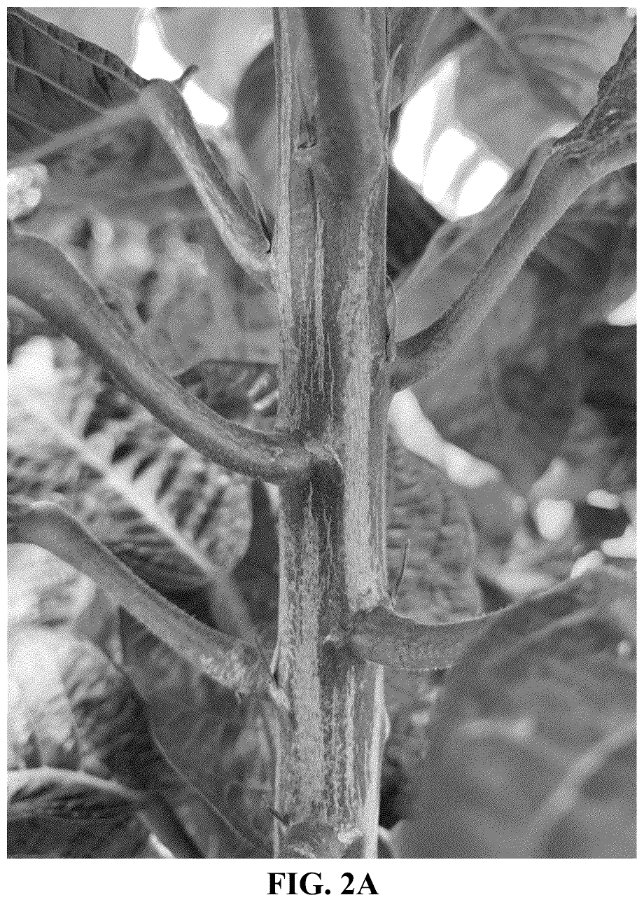

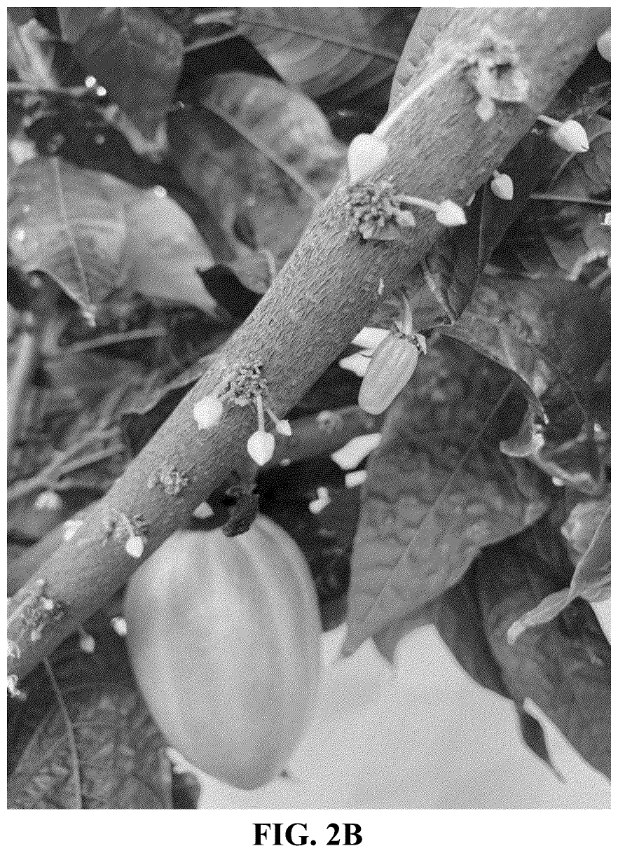

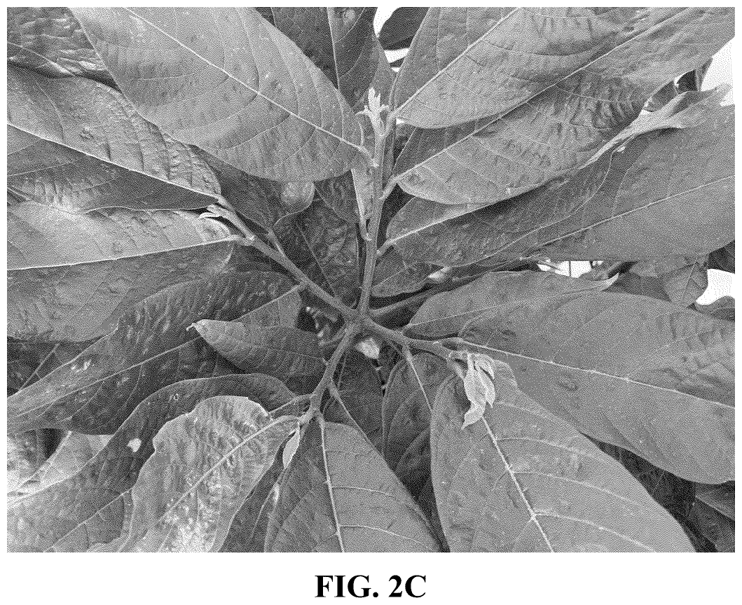

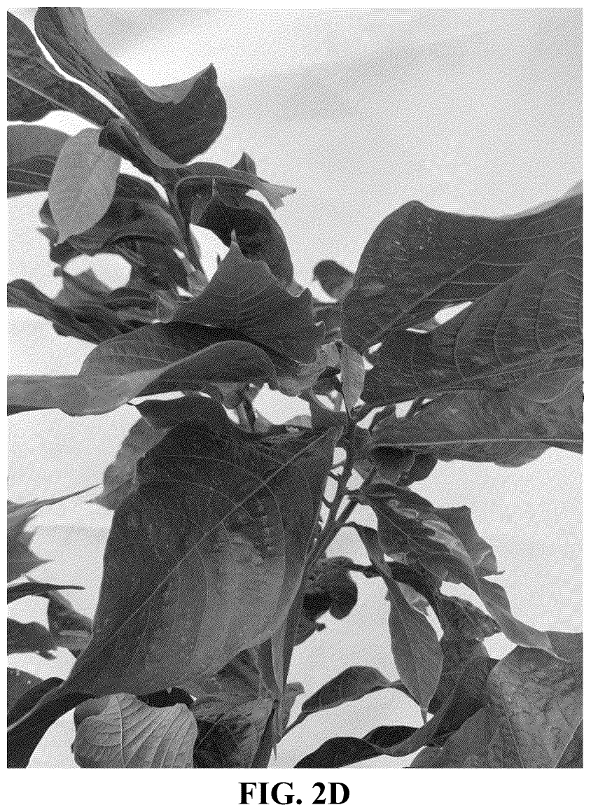

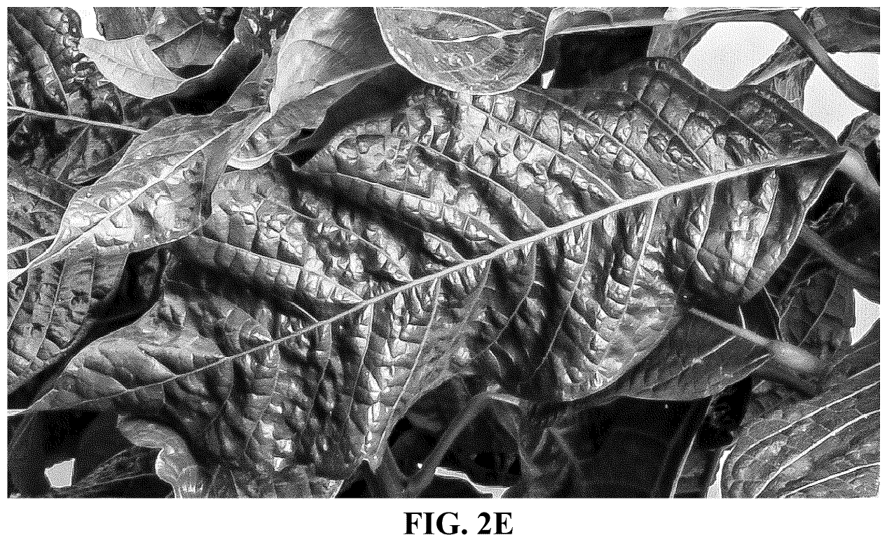

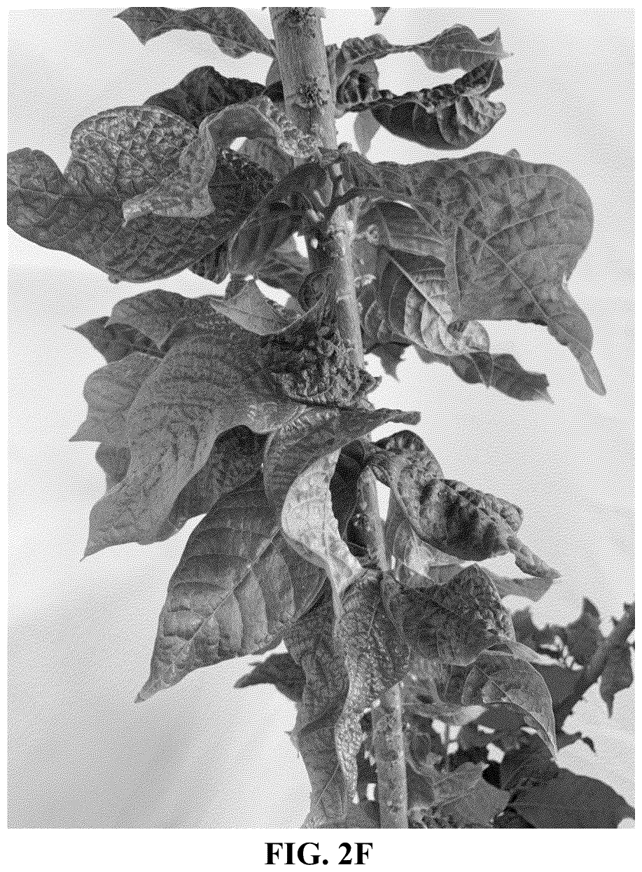

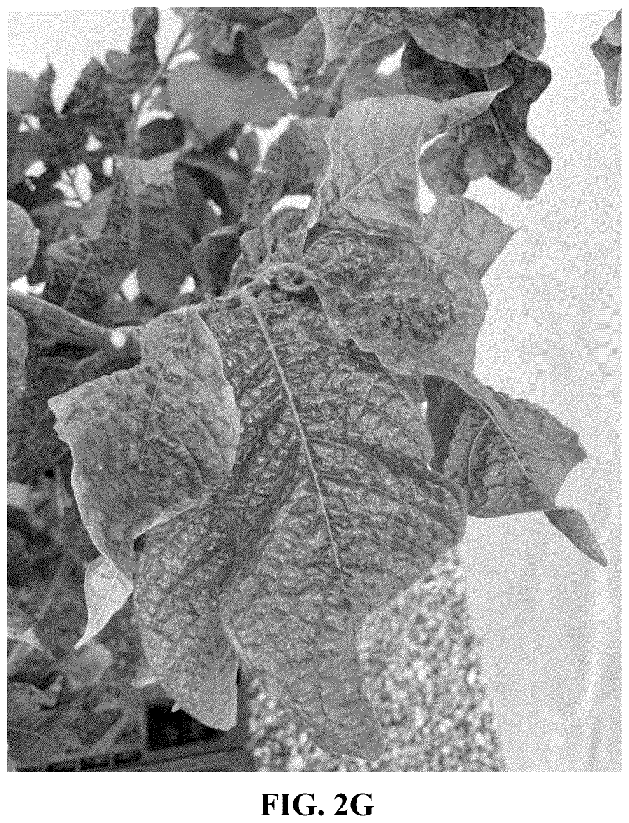

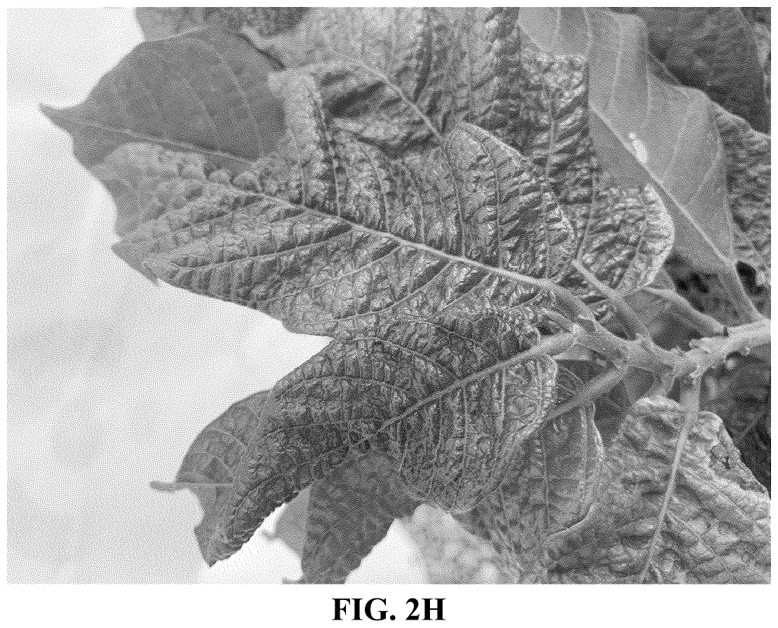

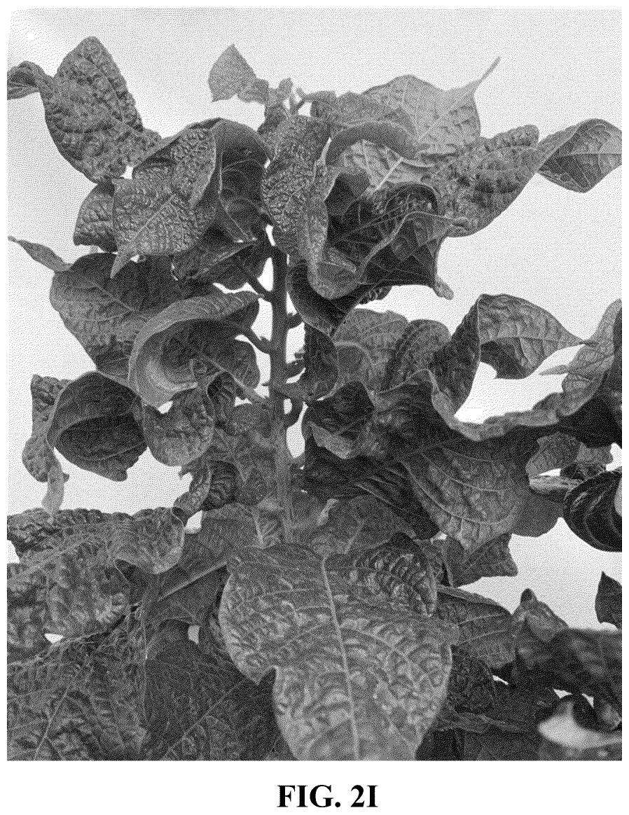

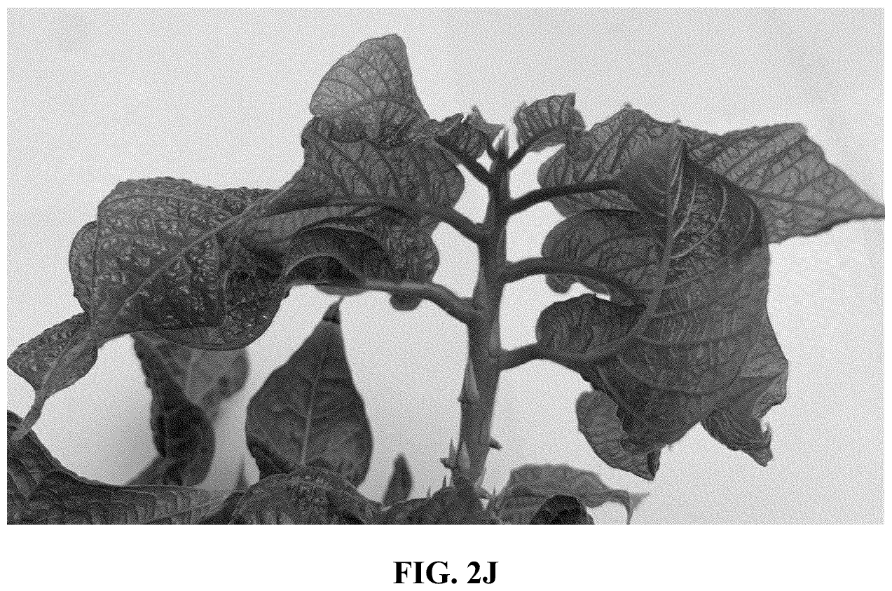

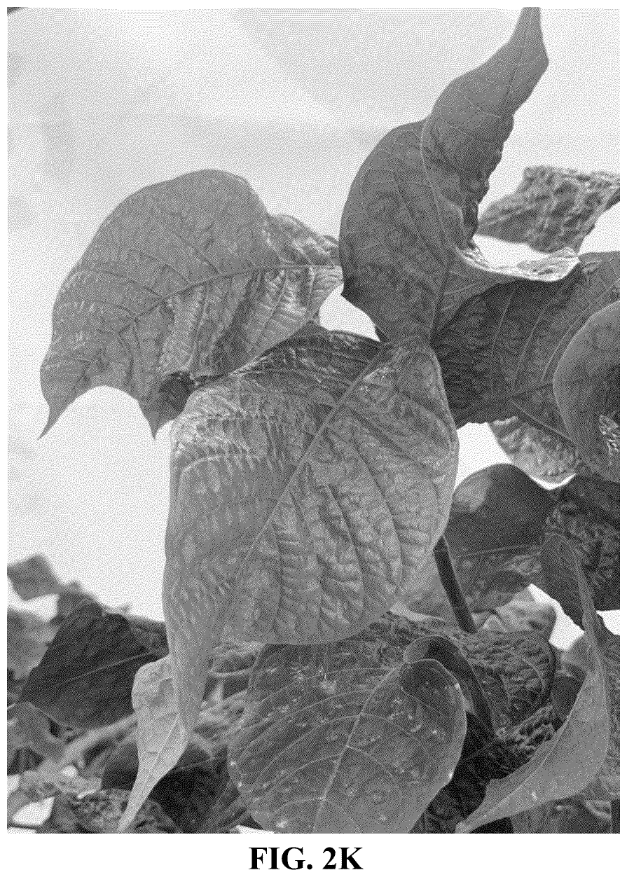

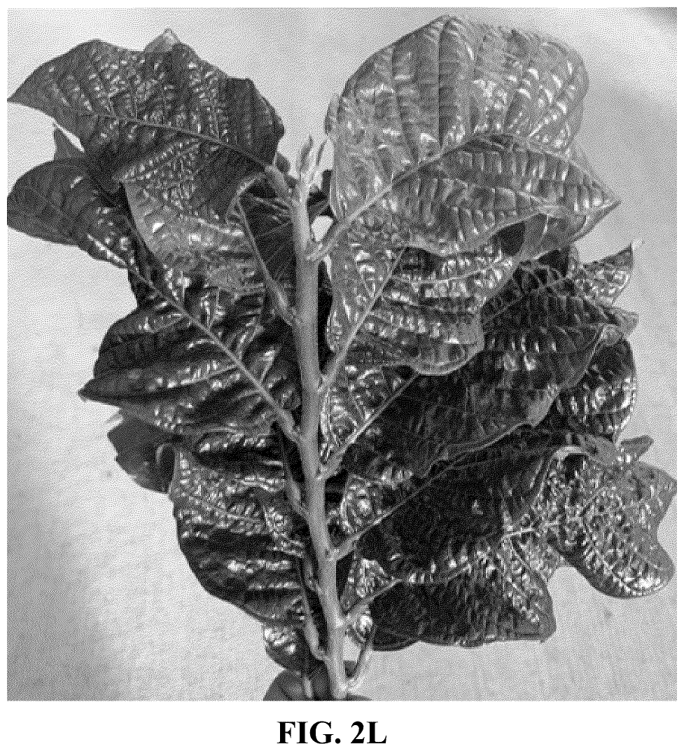

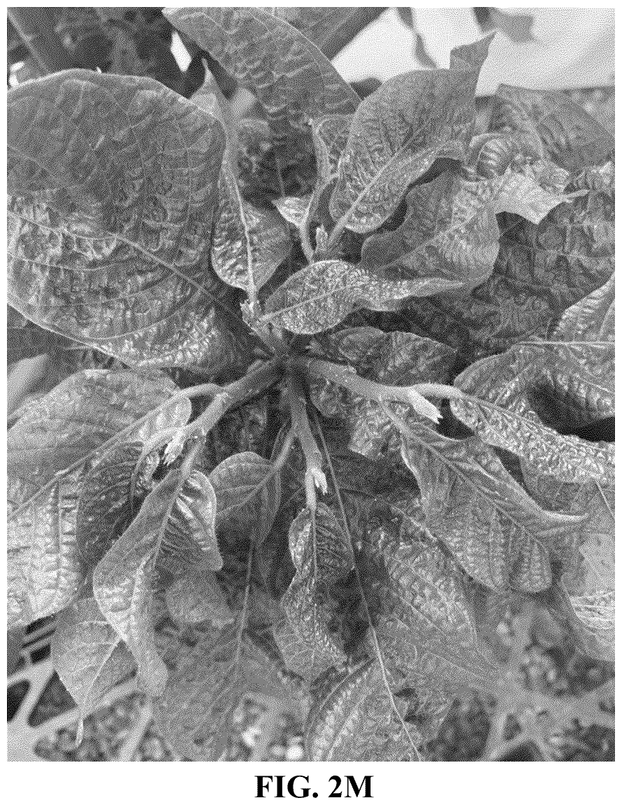

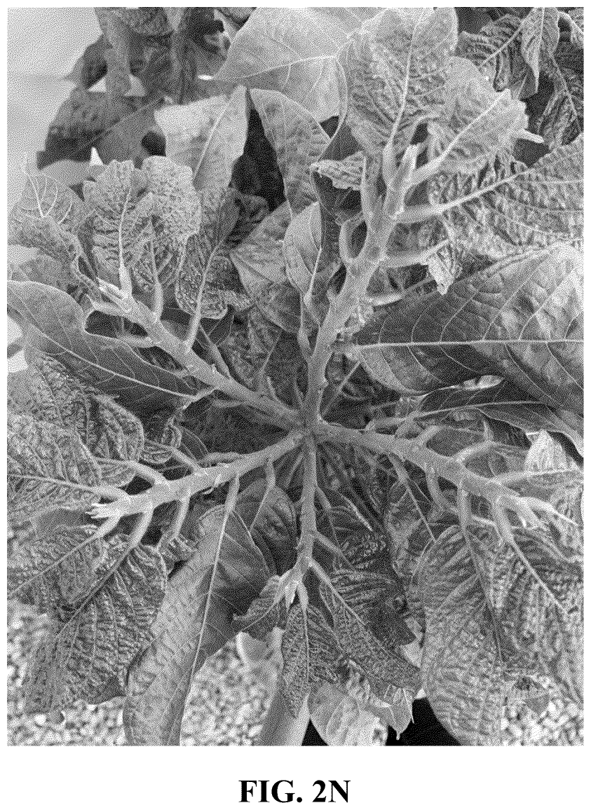

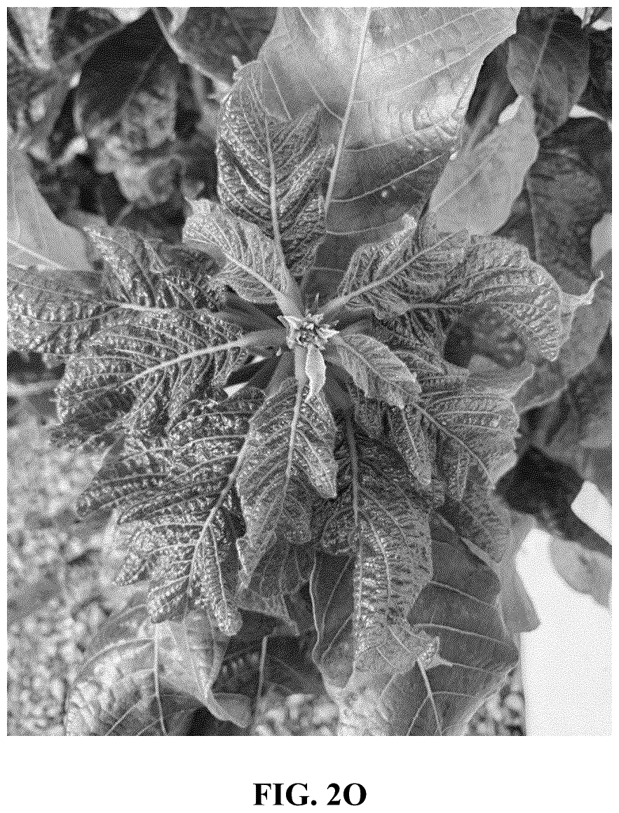

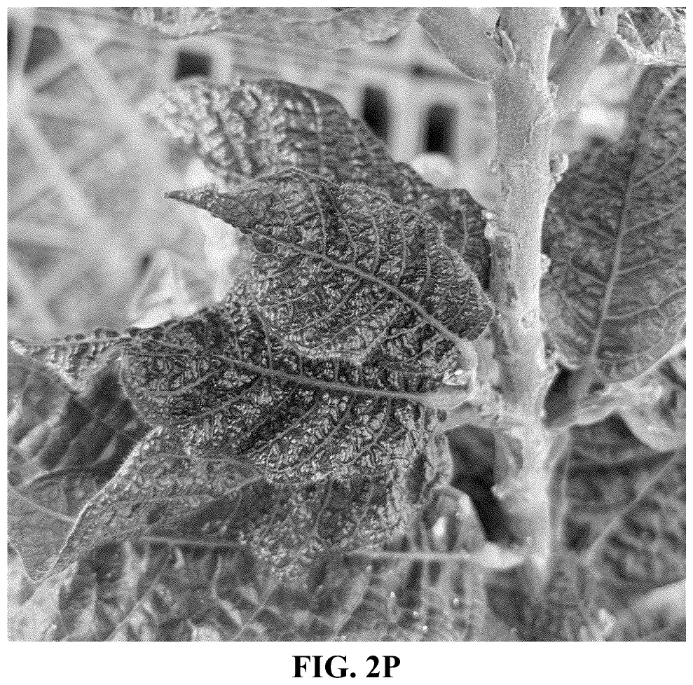

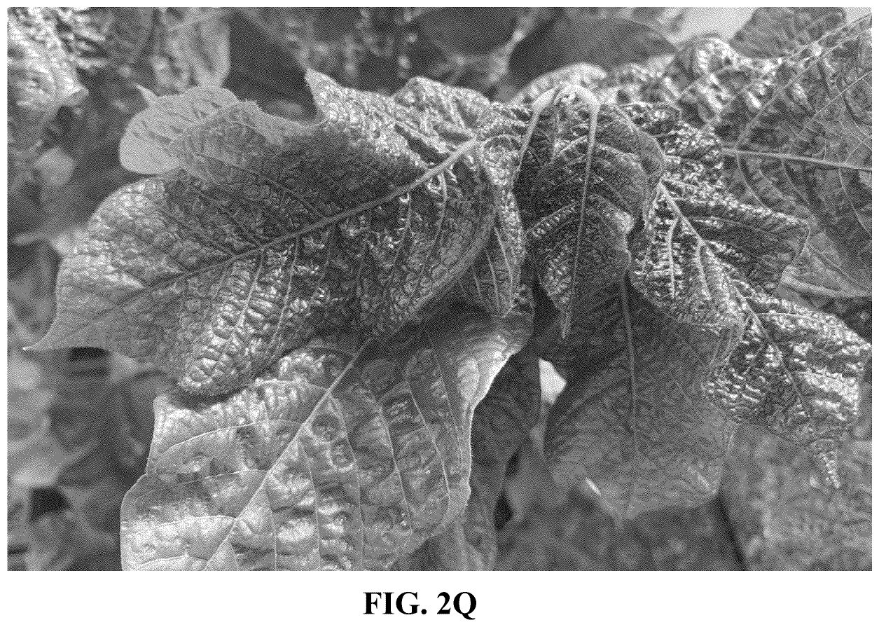

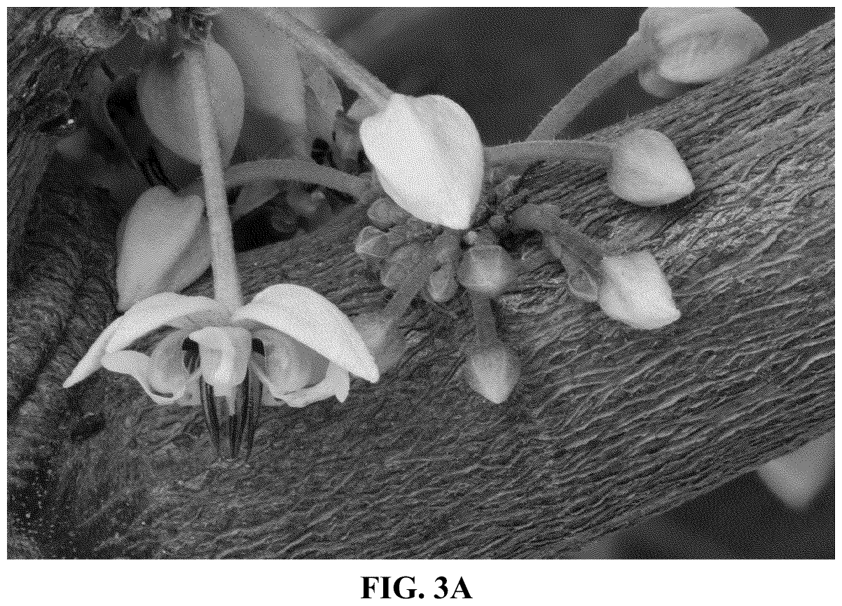

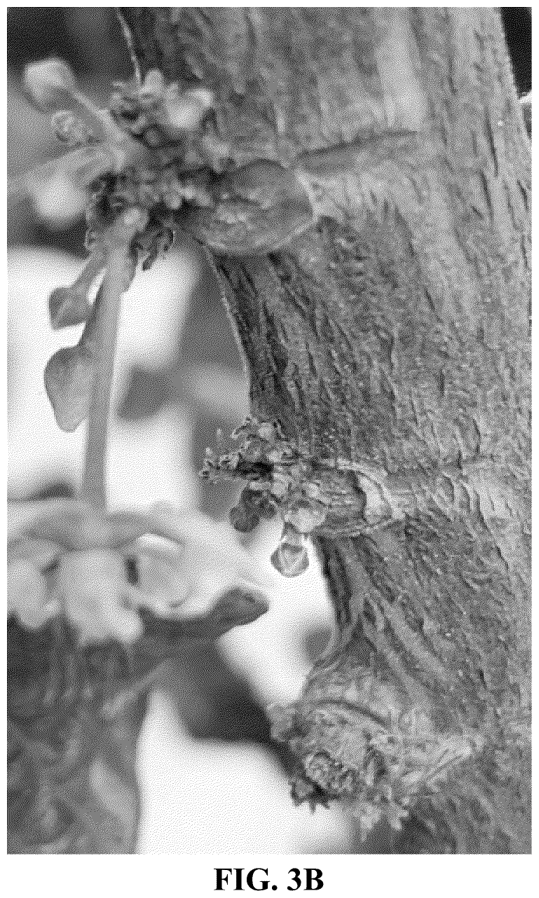

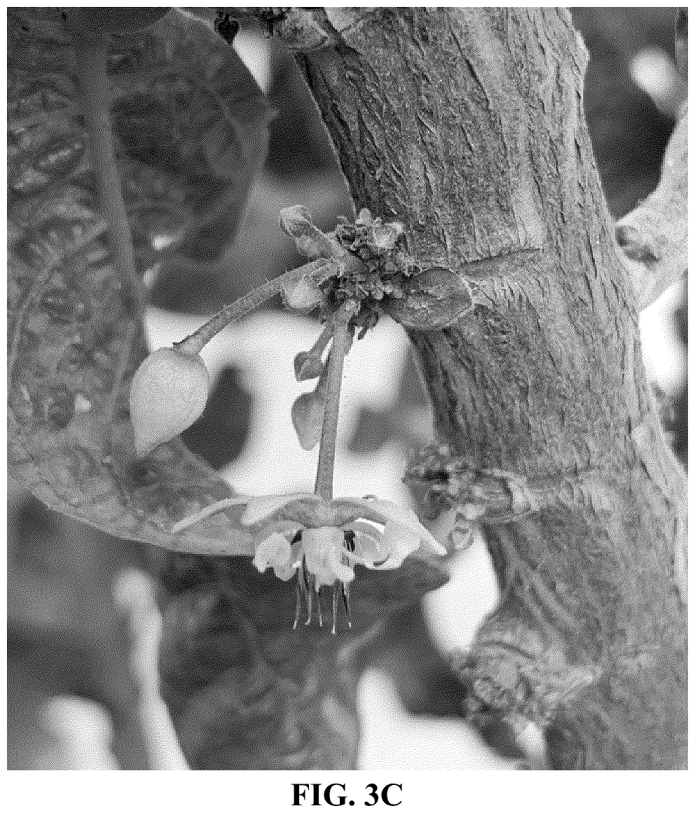

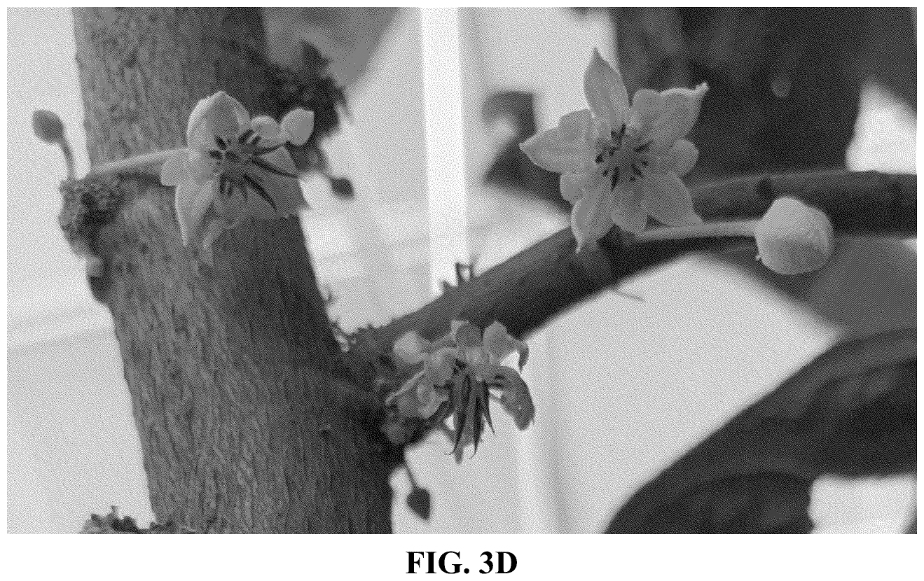

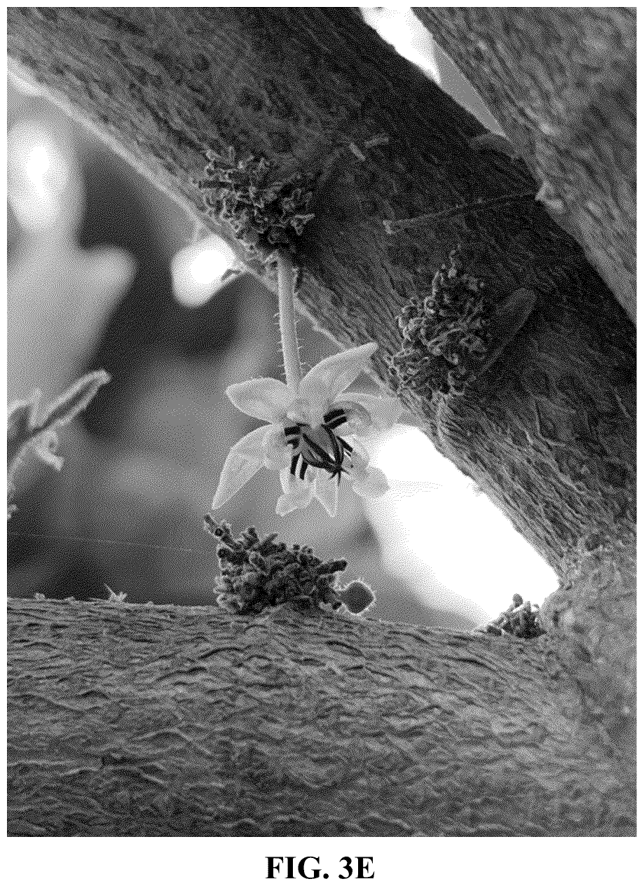

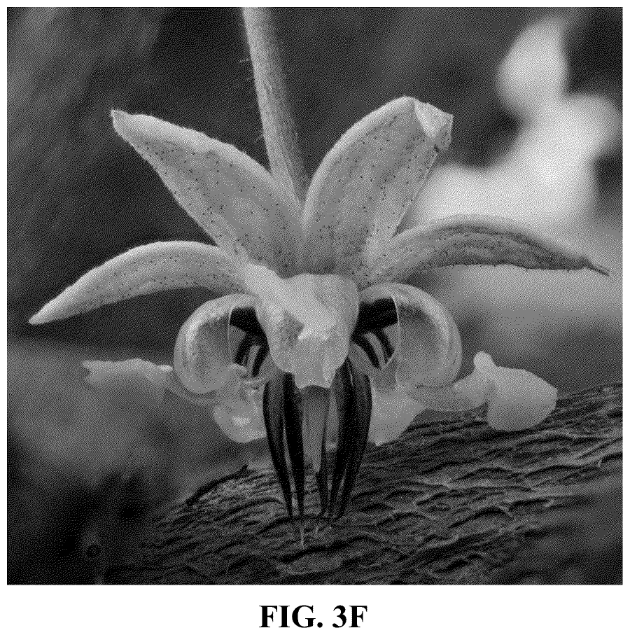

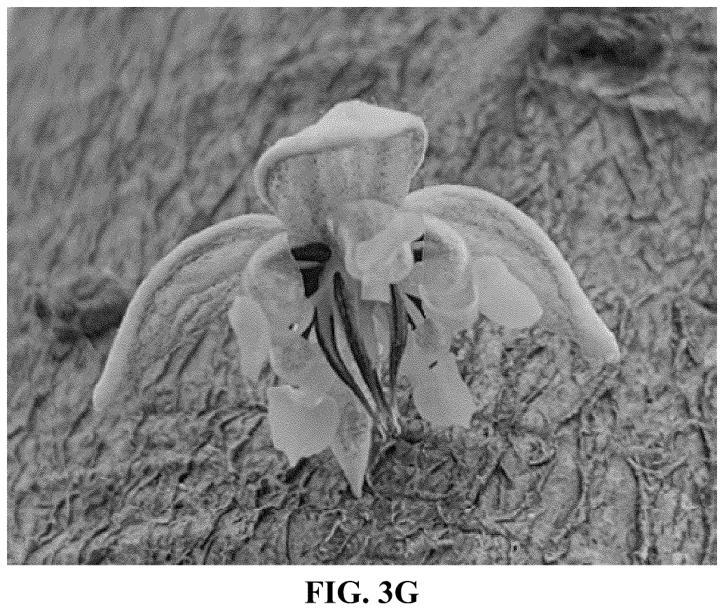

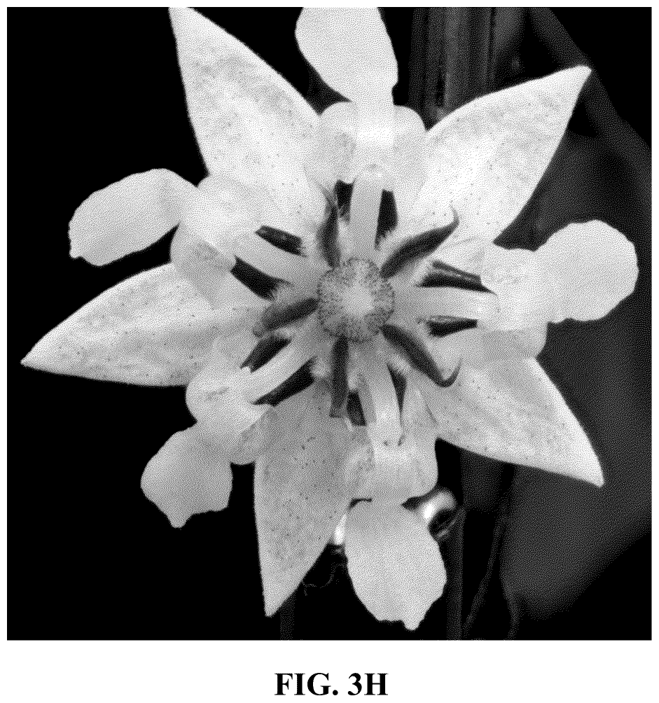

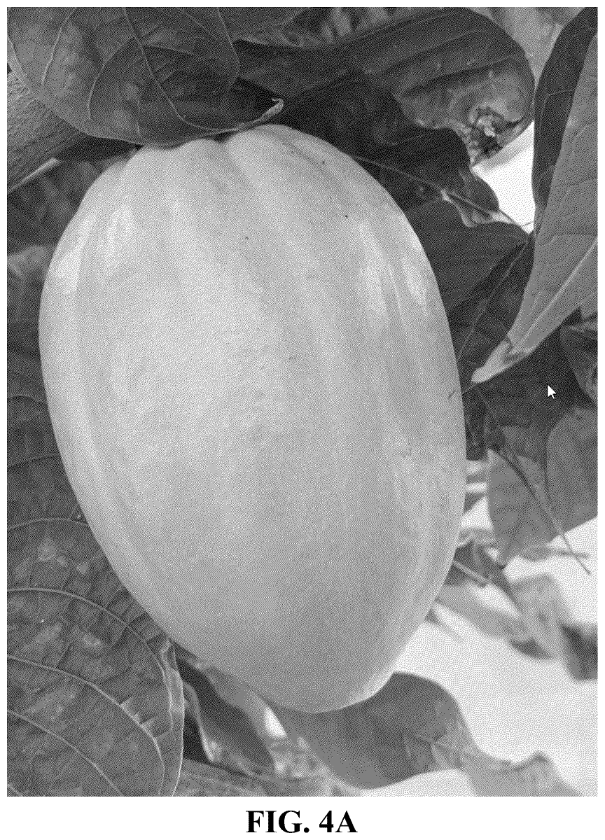

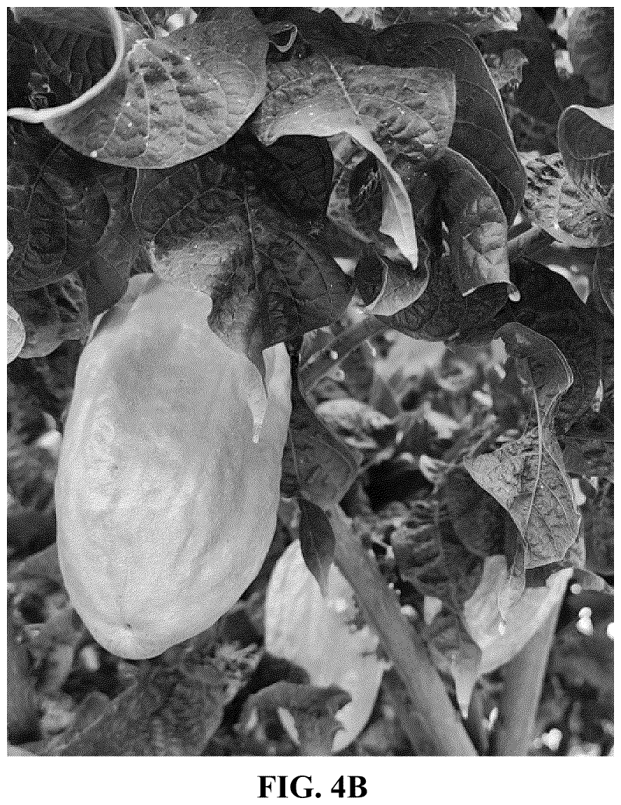

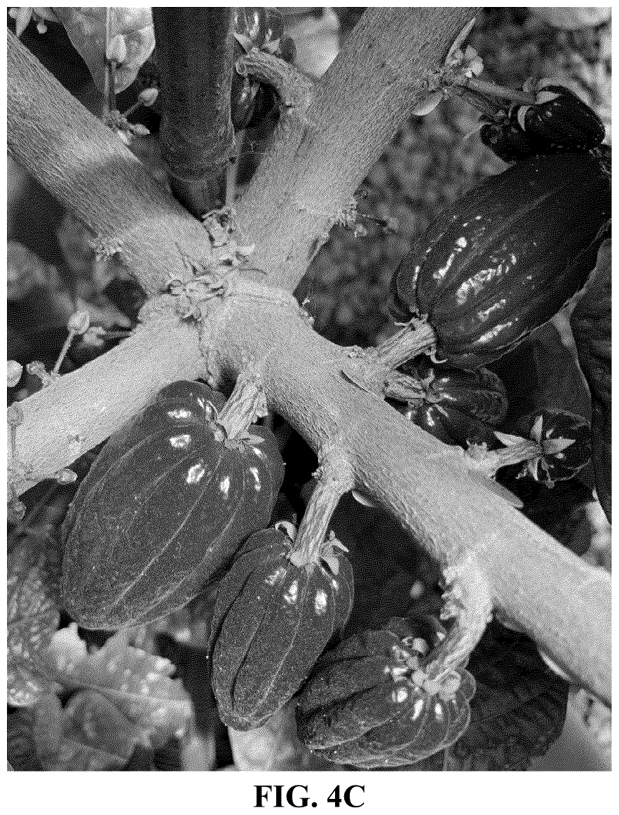

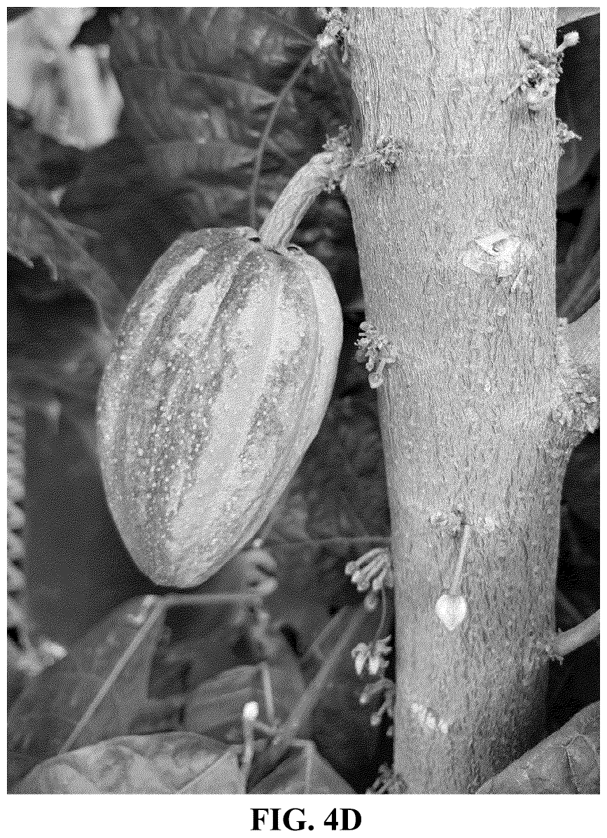

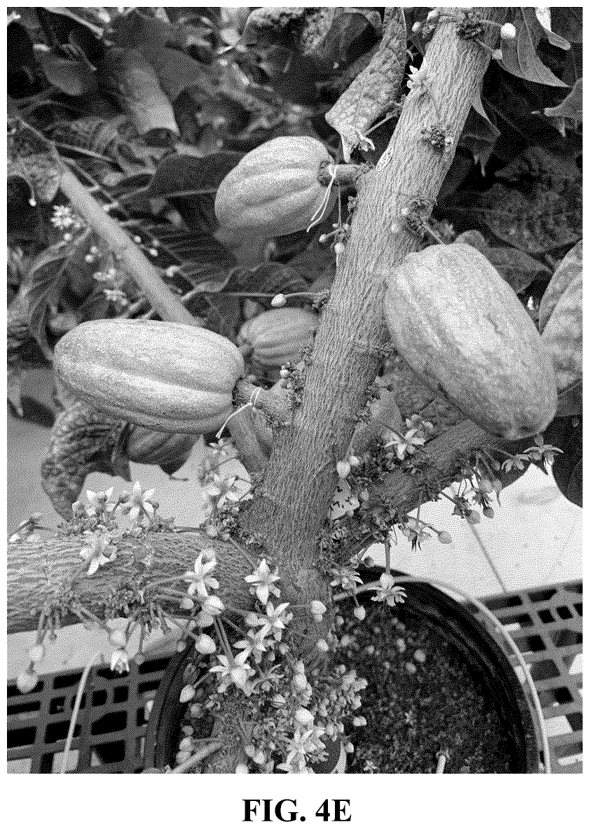

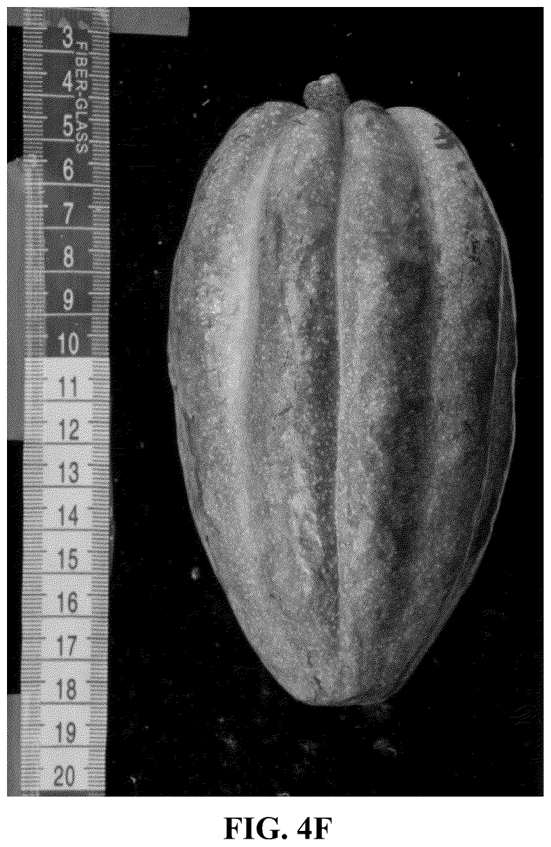

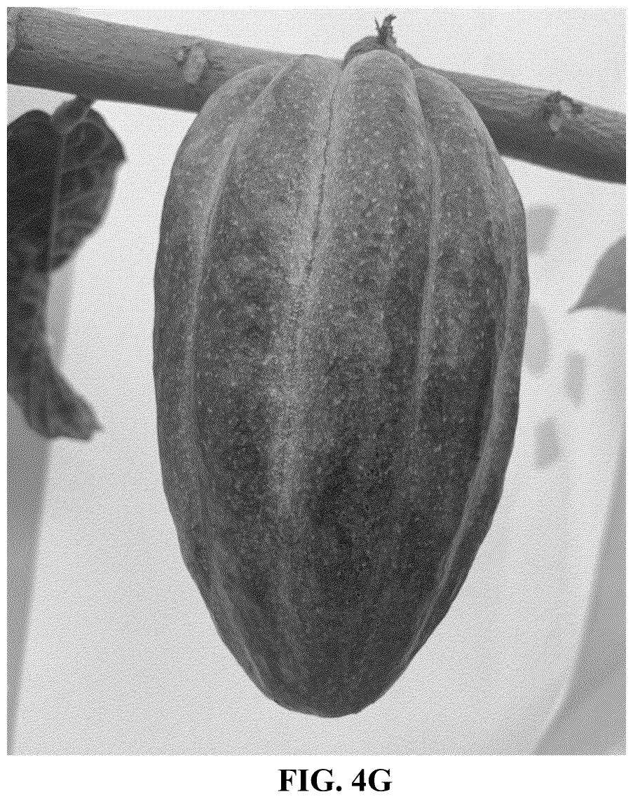

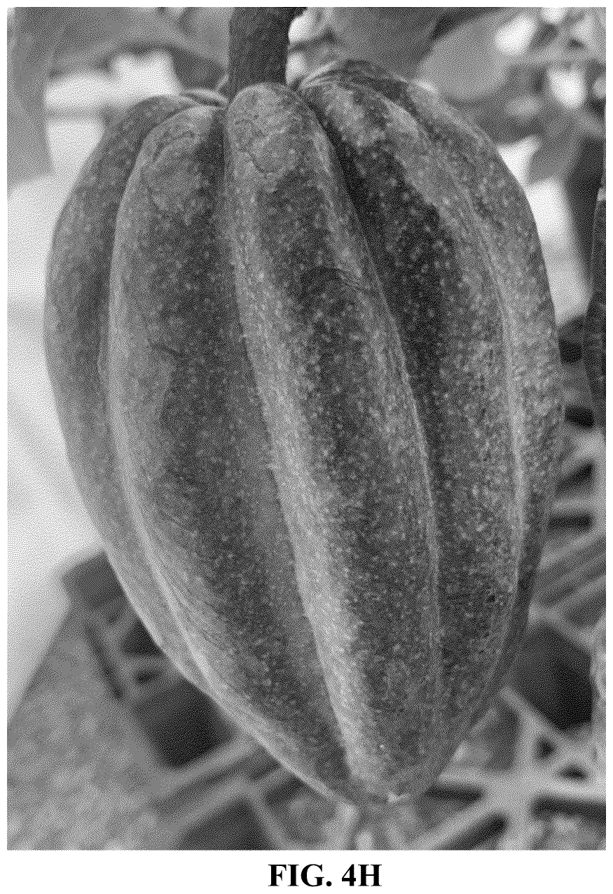

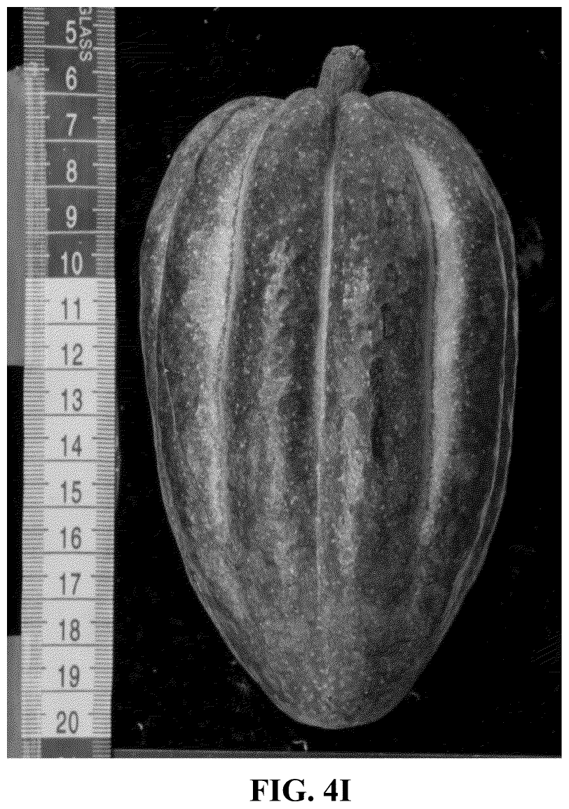

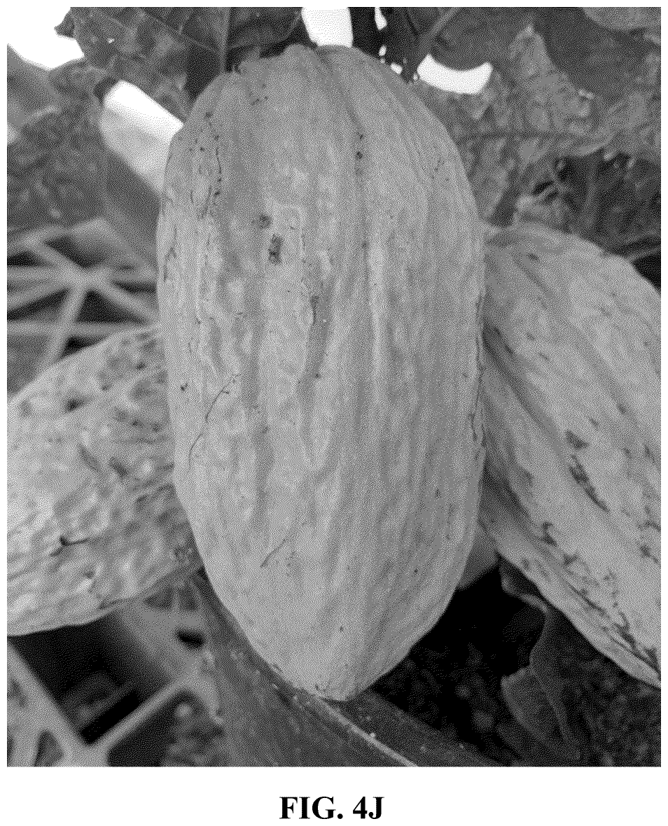

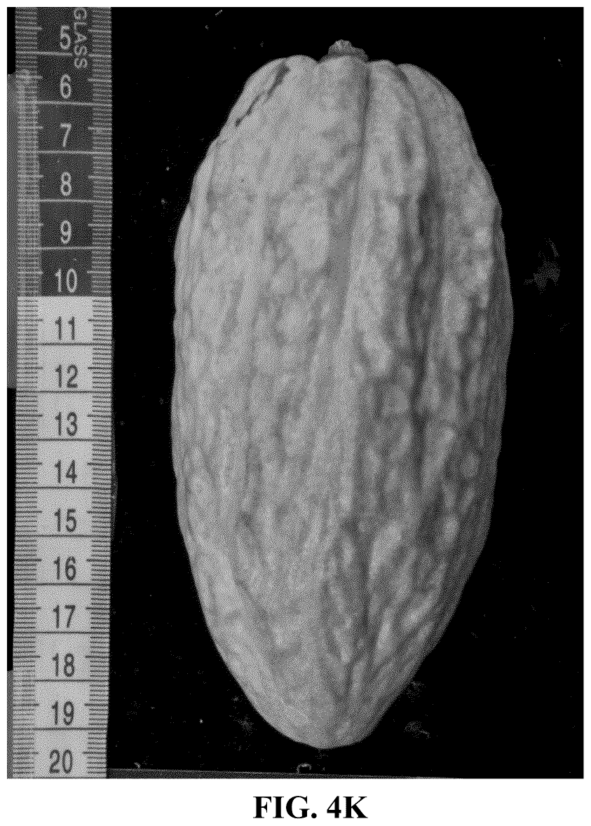

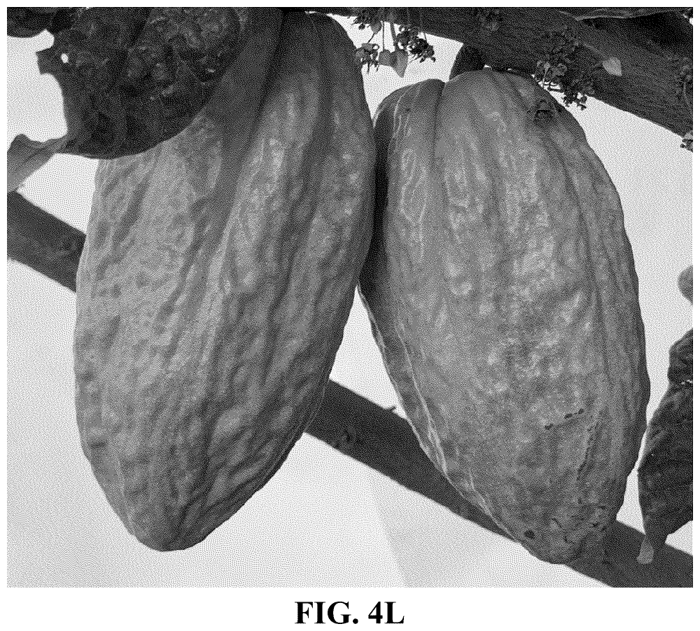

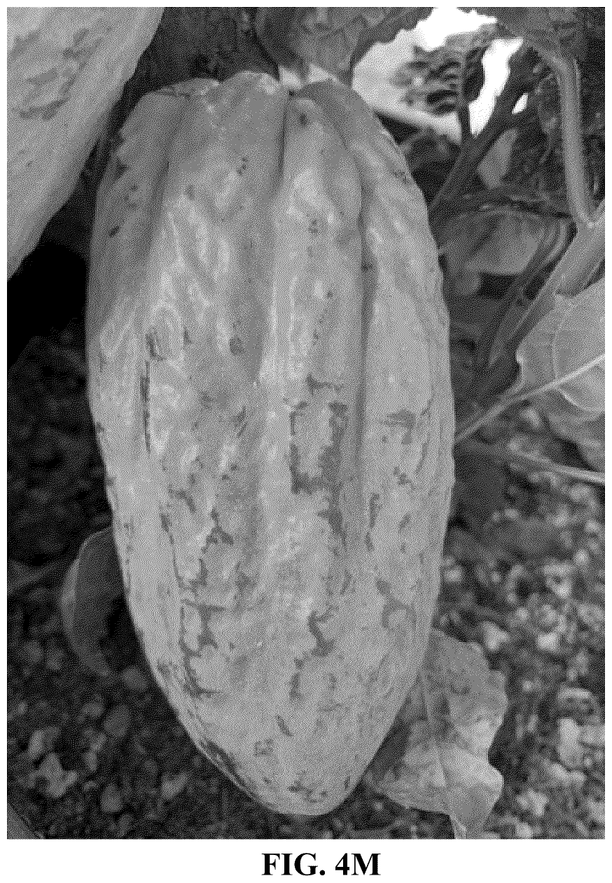

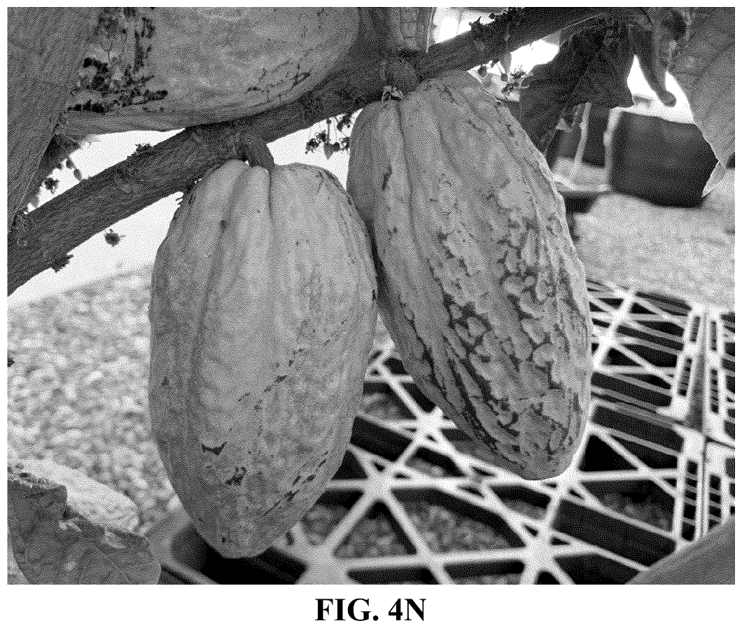

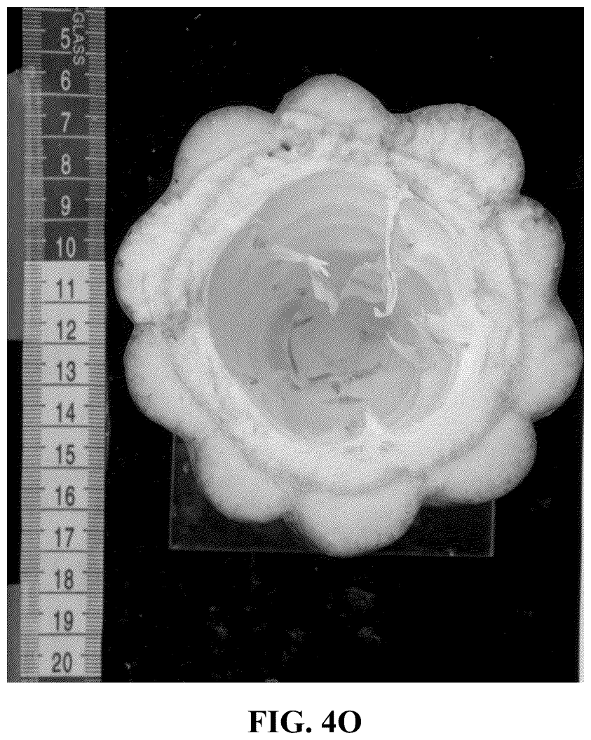

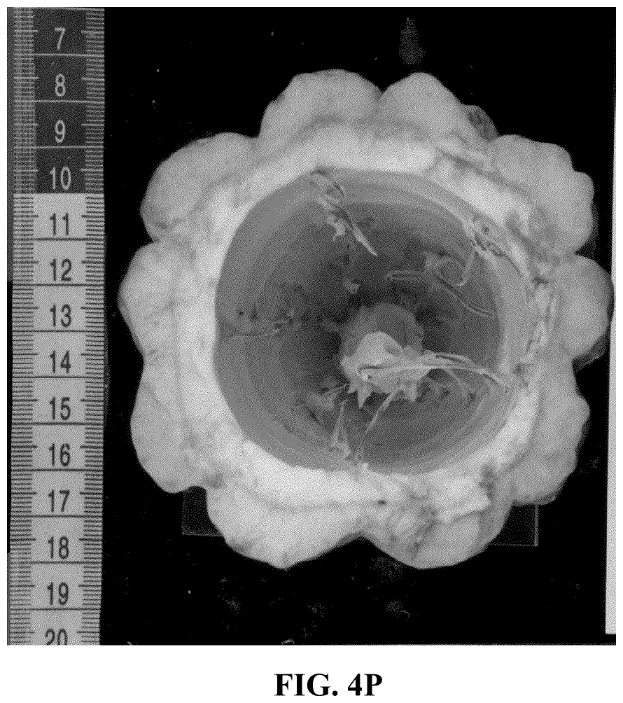

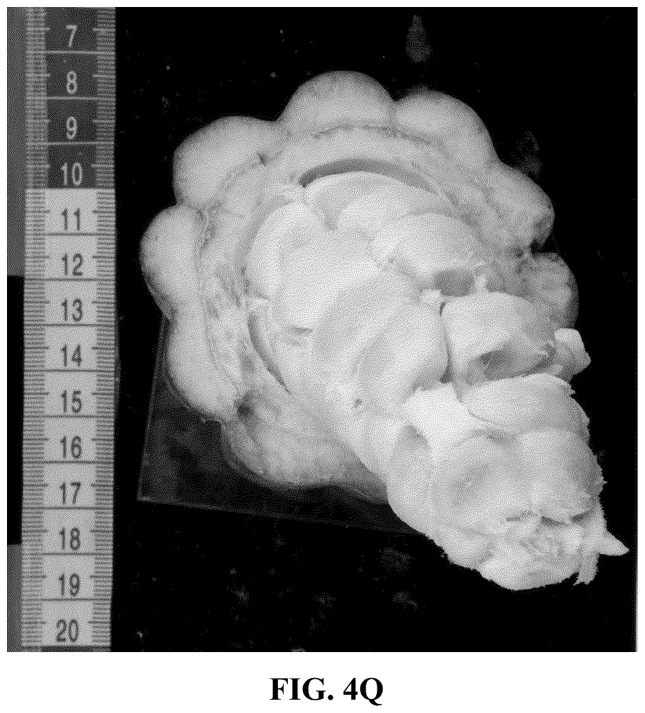

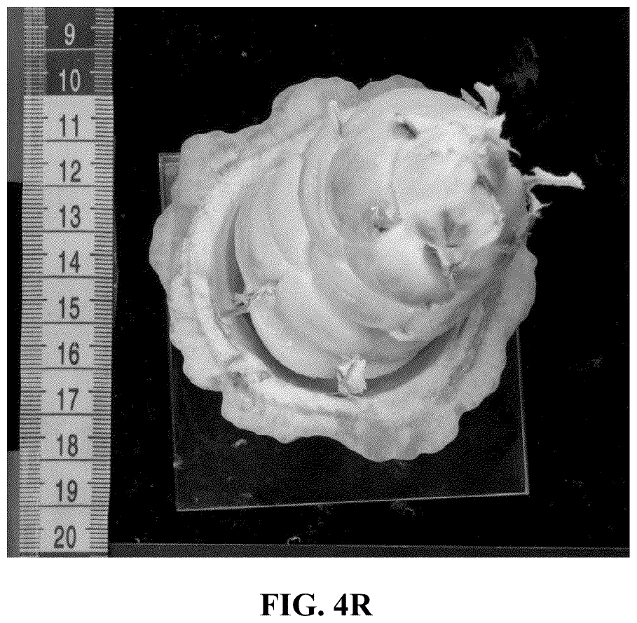

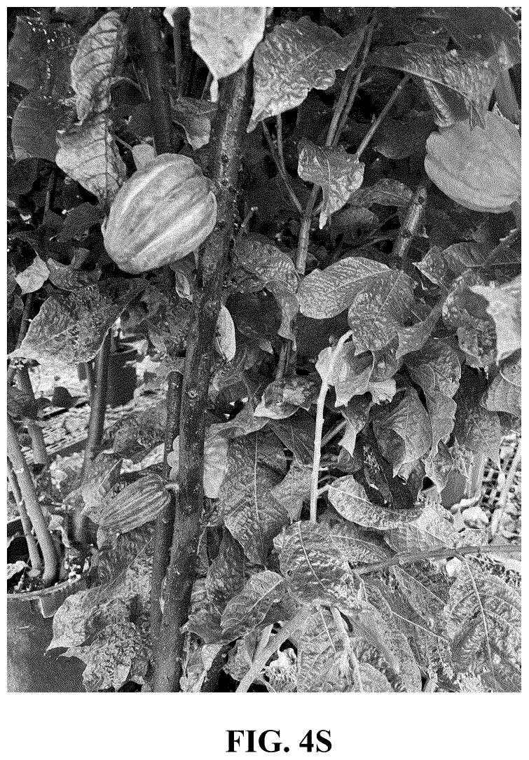

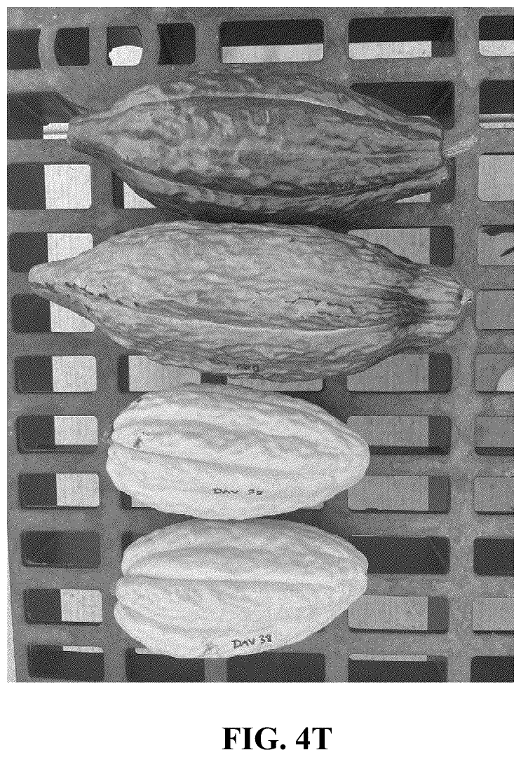

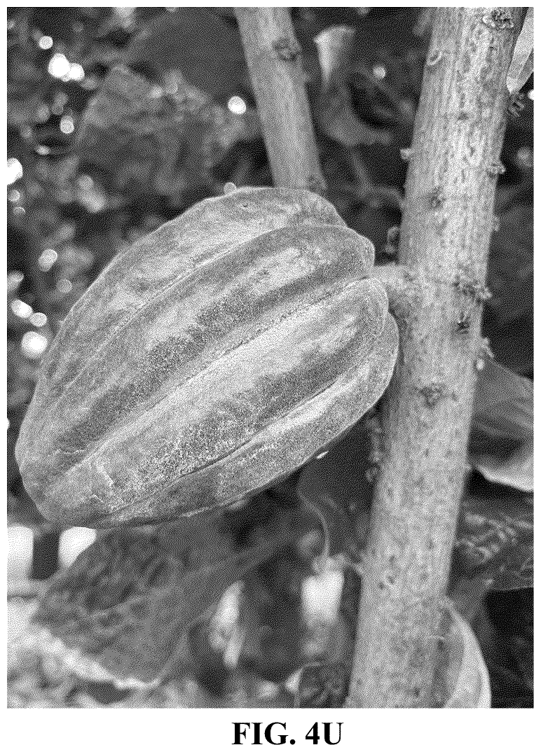

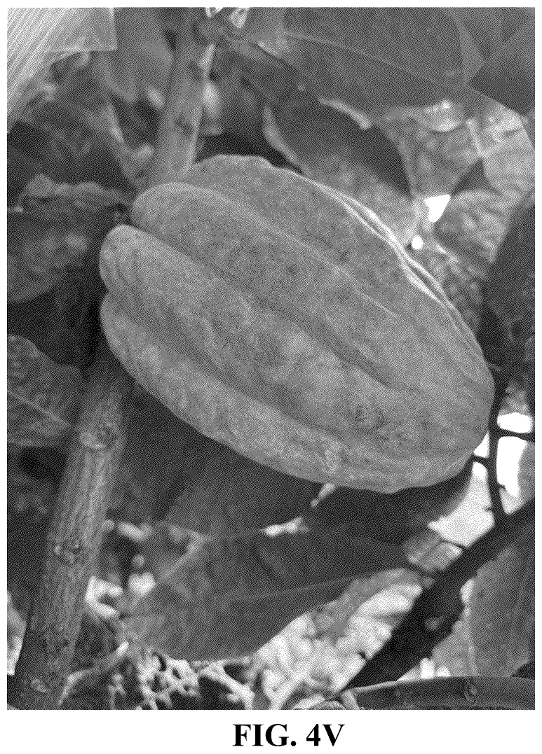

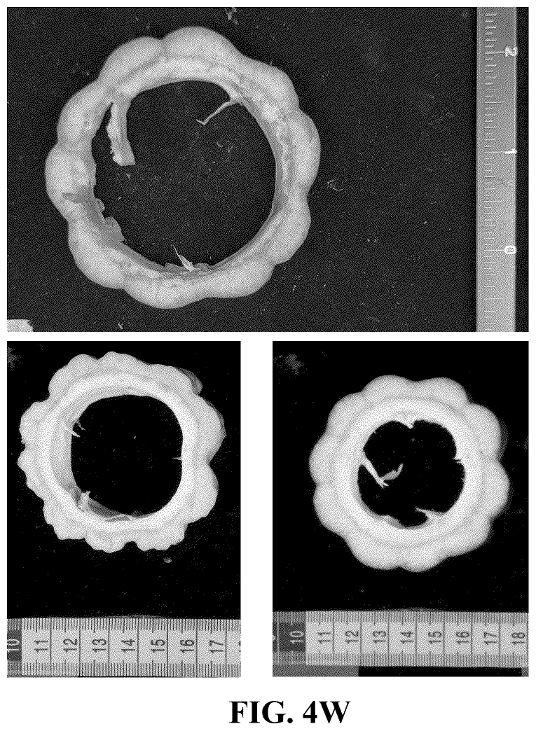

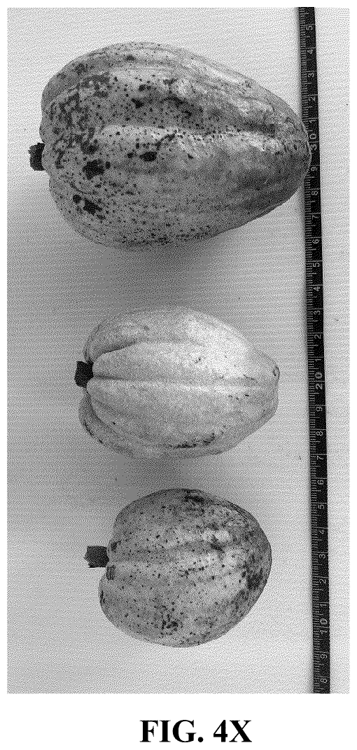

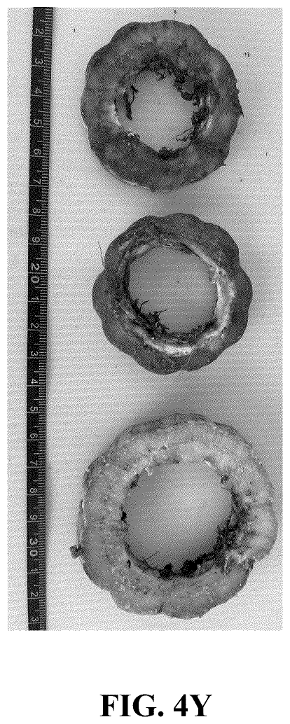

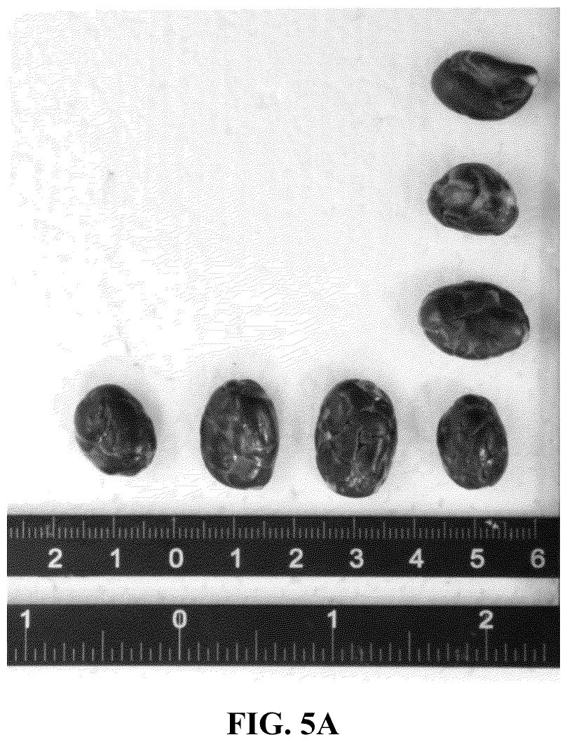

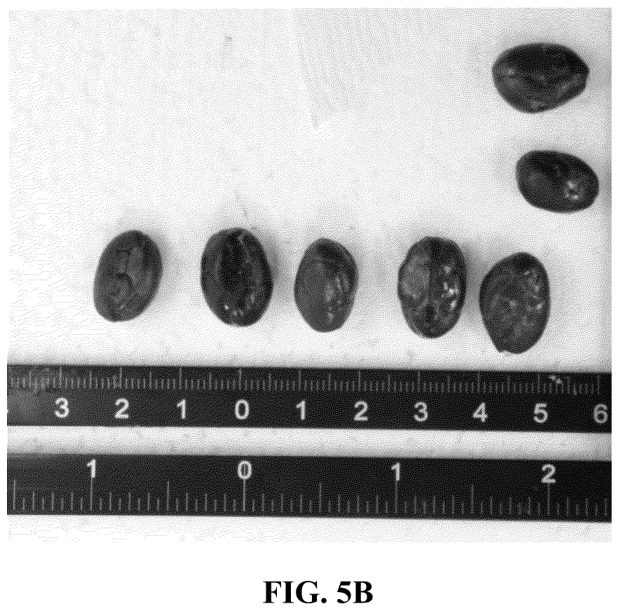

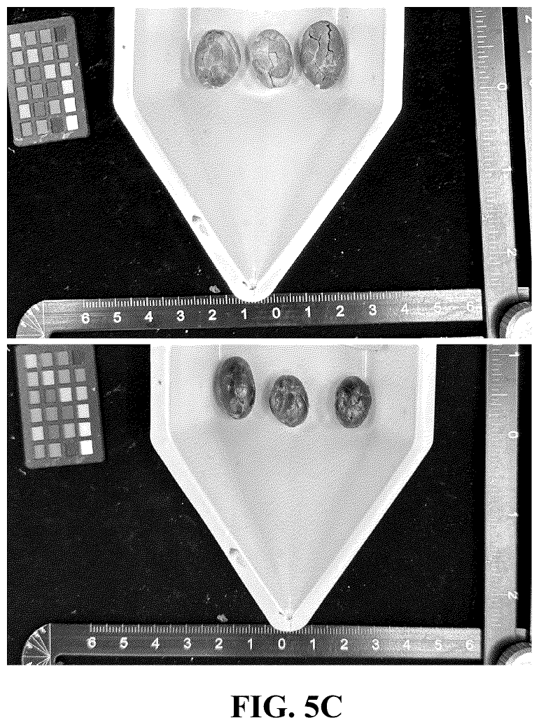

The variety description information in Table 1 provides a summary of variety ‘Mini’ plant characteristics. Those of skill in the art will recognize that these are typical values that may vary due to environment, and that other values that are substantially equivalent are within the scope of the invention. The terminology and descriptors are in line with the descriptors of the “UPOV Guidelines for the Conduct of Tests for Distinctness, Uniformity, and Stability”, or the “Test Guidelines” for Theobroma cacao L. The “Test Guidelines” indicate reference varieties for the descriptors or characteristics that are included in the list. The terminology and descriptors used in these tables are in line with the official terminology as of the filing date, and are thus clear for a person skilled in the art. FIGS. 1-5 illustrate the plant characteristics of variety ‘Mini’.

The following detailed descriptions set forth the distinctive characteristics of ‘Mini’. The data which define these characteristics is based on observations taken in Davis, California, USA. This description is in accordance with UPOV terminology. Color designations, color descriptions, and other phenotypical descriptions may deviate from the stated values and descriptions depending upon variation in environmental, seasonal, climatic, and cultural conditions. Cacao varieties are typically highly susceptible to environmental conditions and often display phenotypic variability among a clonal population based on differences in environmental conditions, such as, for example, based on differences in soil composition. ‘Mini’ has not been observed under all possible environmental conditions. The indicated values represent averages calculated from measurements of several plants. Color references are primarily to The R.H.S. Colour Chart of The Royal Horticultural Society of London (R.H.S.) (2015 edition). Descriptive terminology follows the Plant Identification Terminology, An Illustrated Glossary, 2^{nd} edition by James G. Harris and Melinda Woolf Harris, unless where otherwise defined.

Cacao variety ‘Mini’ is stable and reproduces true to type in successive generations of asexual reproduction within the typical limits of environmental influence in cacao varieties.
Classification:
   Species.—Theobroma cacao L.
   Common name.—Cacao.
   Denomination.—‘Mini’.
Parentage:
   Female parent.—‘CCN-51’.
   Male parent.—‘Crinkle Leaf’.

TABLE 1
Characteristics of cacao variety ‘Mini’

| Characteristic | Value |
|---|---|
| Leaf blade: apex | Acuminate |
| Leaf blade: size | Small |
| Leaf blade: shape of base | Obtuse |
| Leaf blade: intensity of green color | Dark |
| Young leaf: color | Light green to dark red |
| Flower: anthocyanin coloration of pedicel | Absent to strong |
| Flower: length of sepal | Short |
| Flower: width of sepal | Broad |
| Flower: anthocyanin coloration of sepal | Absent to strong |
| Flower: color of ligula | Cream yellow |
| Flower: anthocyanin coloration of sepal | absent to moderate |
| Fruit: shape | Oblong, obovate or elliptic |
| Fruit: basal constriction | Absent to strong |
| Fruit: shape of apex | Acute, obtuse or rounded |
| Fruit: length | Medium |
| Fruit: diameter | Small to medium |
| Fruit: length/diameter ratio | Medium to moderately elongated |
| Fruit: depth of grooves | Shallow to deep |
| Fruit: color | Green, yellow, medium red, dark red |
| Fruit: exocarp thickness | Thin to medium |
| Fruit: color of pulp | Light cream |
| Fruit: sweetness of pulp | medium |
| Fruit: number of seeds | Medium to many |
| Seed: shape in longitudinal section | Oblong or elliptic |
| Seed: length | Small to medium |
| Seed: width | Medium to broad |
| Seed: ratio length/width | Moderately compressed to moderately |
| | elongated |
| Seed: thickness | Thin to medium |
| Seed: cotyledon color | Pink to dark purple |

### COMPARISON WITH PARENTAL AND REFERENCE VARIETIES

Cacao variety ‘Crinkle Leaf’ (unpatented) is the paternal clone used to develop variety ‘Mini’ and may be used as a comparison variety for cacao variety ‘Mini’ as described herein, including as described in Table 2 and Table 3 below.

TABLE 2
Comparative characteristics between
variety ‘Mini’ and variety ‘Crinkle Leaf’

| Characteristic | Expression | |
|---|---|---|
| in which | of the | Expression |
| variety ‘Mini’ | characteristic | of the |
| differs variety | in variety | characteristic |
| ‘Crinkle Leaf’ | ‘Crinkle Leaf’ | in variety ‘Mini’ |
| Fruit: color | Orange | Green, yellow, |
| | | medium red, |
| | | dark red |
| Fruit: length | Short | Medium to long |
| Fruit: diameter | Small | Medium |
| Fruit: length/ | Moderately | Medium to |
| diameter ratio | compressed | moderately |
| | | elongated |
| Fruit: shape | Circular to ovate | Elliptic to oblong |
| Fruit: surface | Smooth | Smooth to rough |
| Fruit: number of seeds | Few | Medium to many |
| Fruit: exocarp thickness | Thick | Thin to medium |
| Fruit: depth of grooves | Absent or | Shallow to deep |
| | very shallow | |
| Young leaf: color | Light red | Light green |
| | | to dark red |
| Flower: anthocyanin | Moderate | Absent to |
| of pedicel | | moderate |
| Flower: length of sepal | Short | Medium |
| Flower: width of sepal | Short | Medium |

Cacao variety ‘Mini’ is derived from the parent ‘Crinkle Leaf’ but differs in several characteristics, most notably in characteristics of the flushes and pods (Table 2). Pods of Cacao variety ‘Mini’ are longer, wider, and bear more seeds than those of variety ‘Crinkle Leaf’ (Table 3). Pods of variety ‘Mini’ also vary in pigmentation at maturity between yellow, orange, and red, while those of parent variety ‘Crinkle Leaf’ are orange at maturation. Surface texture is distinct as well, with surface rugosity ranging from absent to intermediate in cacao variety ‘Mini’, and only absent in variety ‘Crinkle Leaf’.

Furthermore, cacao variety ‘Mini’ may be characterized by deep furrows, while ‘Crinkle Leaf’ is not. Floral parts including sepal, pedicel, ligule and staminodes and ovaries are larger in cacao variety ‘Mini’ than in ‘Crinkle Leaf’.

TABLE 3
Pod traits of variety ‘Mini’ compared to variety ‘Crinkle Leaf’

| | | Number | Pod | Pod |
|---|---|---|---|---|
| Variety | Pod ID | of Seeds | Circumference | Length |
| ‘Mini’ | 41-2 | 44 | 25.8 | 13.8 |
| ‘Mini’ | 51-1 | 23 | 22.2 | 14.4 |
| ‘Mini’ | 51-2 | 22 | 20.9 | 12 |
| ‘Mini’ | 53-1 | 20 | 23 | 8.8 |
| ‘Mini’ | 56-1 | 45 | 25 | 12.3 |
| ‘Mini’ | 56-2 | 31 | 23.6 | 10.1 |
| ‘Mini’ | 59-1 | 14 | 19.3 | 11 |
| ‘Mini’ | 59-2 | 38 | 23.2 | 12.8 |
| ‘Crinkle Leaf’ | 292-1 | 6 | 7.5 | 6.5 |
| ‘Crinkle Leaf’ | 292-2 | 10 | 8.5 | 8 |
| ‘Crinkle Leaf’ | 292-3 | 19 | 23.25 | 9.5 |

We claim:

1. A new and distinct variety of cacao plant named ‘Mini’ as shown and described herein.
